(19) United States
(12) Patent Application Publication
(10) Pub. No.: US 2025/0072873 A1
(43) Pub. Date: Mar. 6, 2025

(54) METHODS AND SYSTEMS FOR ULTRASOUND IMAGING

(71) Applicant: WUHAN UNITED IMAGING HEALTHCARE CO., LTD., Wuhan (CN)

(72) Inventors: Aiwei SHI, Wuhan (CN); Jianmin ZHANG, Wuhan (CN)

(73) Assignee: WUHAN UNITED IMAGING HEALTHCARE CO., LTD., Wuhan (CN)

(21) Appl. No.: 18/822,753

(22) Filed: Sep. 3, 2024

(30) Foreign Application Priority Data
Sep. 4, 2023 (CN) 202311134722.1

(51) Int. Cl. A61B 8/08

(52) U.S. Cl. CPC A61B 8/5269

(57) ABSTRACT

The present disclosure provides a method and a system for ultrasound imaging. The method comprises determining a weight dataset corresponding to each original image, the weight dataset including first weight information, the first weight information including a plurality of pieces of first weight information, counts of the plurality of pieces of first weight information for the plurality of original images being the same; for each weight data subset in the weight dataset, determining a composite sub-image corresponding to the each weight data subset based on a plurality of original images and a plurality of pieces of first weight information corresponding to the each weight data subset, and determining a target image based on a plurality of composite sub-images corresponding to a plurality of weight data subsets in the weight dataset.

### CROSS-REFERENCE TO RELATED APPLICATIONS

The present disclosure claims priority to Chinese application No. 202311134722.1, filed on Sep. 4, 2023, the entire contents of which are hereby incorporated herein by reference.

### TECHNICAL FIELD

The present disclosure relates to the field of ultrasound imaging, and in particular, to a method and a system for ultrasound imaging.

### BACKGROUND

Due to the physical properties of ultrasound, speckle noise inevitably exists in ultrasound medical imaging. In order to reduce the speckle noise, the methods usually used are frequency domain composition and spatial composition. Frequency domain composition is usually to decompose echo signals into different channels or transmit ultrasound of different frequencies, so as to obtain the data formed by different frequency bands, and by superimposing these data, the purpose of removing speckle noise can be achieved.

Spatial composition typically involves emitting ultrasound beams at different emission angles and utilizing the superposition of their echo signals to eliminate speckle noise. Frequency domain composition can be limited in its effectiveness by the signal and system bandwidth, making spatial composition a more widely adopted and effective method of eliminating speckle noise. However, conventional spatial composition methods require the emission of acoustic waves at different angles, which, for fast-moving tissues, significantly reduces the resolution of the image and may even result in image trailing.

Therefore, a method and a system for ultrasound imaging are provided, in which a plurality of ultrasound images are obtained with a single emission by performing a weighted processing on the ultrasound images through designing different weights, which is capable of effectively eliminating speckle noise of a target ultrasound image and improving a signal-to-noise ratio and resolution of the target ultrasound image while avoiding artifacts and spatial resolution degradation caused by tissue motion.

### SUMMARY

One or more embodiments of the present disclosure provide a method for ultrasound imaging, implemented on a computing device having at least one processor and at least one storage device, the method comprising: obtaining a plurality of original images produced based on a plurality of emissions of ultrasound waves, emission angles, or emission positions corresponding to the plurality of emissions of ultrasound waves being different; for each original image of the plurality of original images, determining a weight dataset corresponding to the each original image, the weight dataset including a plurality of weight data subsets, a count of the plurality of weight data subsets for each of the plurality of original images being the same; for each weight data subset in the weight dataset, determining a composite sub-image corresponding to the each weight data subset based on the plurality of original images and a plurality of pieces of first weight information corresponding to the each weight data subset; and determining a target image based on a plurality of composite sub-images corresponding to the plurality of weight data subsets in the weight dataset.

In some embodiments, at least two pieces of first weight information corresponding to a same weight data subset among at least two of the plurality of weight datasets are the same.

In some embodiments, the first weight information corresponding to the each original image includes a weight value corresponding to each position in the each original image.

In some embodiments, the for each weight data subset in the weight dataset, determining a composite sub-image corresponding to the each weight data subset based on the plurality of original images and a plurality of pieces of first weight information corresponding to the each weight data subset includes: for each weight data subset in the weight dataset, determining a plurality of weighted original images based on the plurality of original images and the plurality of pieces of first weight information corresponding to the each weight data subset; and determining the composite sub-image corresponding to the each weight data subset by performing coherent compounding on the plurality of weighted original images.

In some embodiments, the determining a target image based on a plurality of composite sub-images corresponding to the plurality of weight data subsets in the weight dataset includes: generating a processed composite sub-image by performing an envelope detection processing on each composite sub-image; for the each composite sub-image, determining second weight information by performing quality analysis on the each composite sub-image or the processed composite sub-image corresponding to the each composite sub-image; and generating the target image based on the processed composite sub-image and the second weight information corresponding to the each composite sub-image.

In some embodiments, the first weight information is denoted by a first weight image, the first weight image including one or more weighted regions, and weighted regions of different first weight images being different.

In some embodiments, in response to the ultrasound waves being wide beams, the first weight information is determined such that each of the plurality of composite sub-images is a complete image, or a combination of the plurality of composite sub-images is a complete image.

In some embodiments, the first weight information is denoted by a first weight image, the first weight image including one or more weighted regions, the one or more weighted regions including a high-weighted region, different high-weighted regions in different first weight images have different positions or widths in a lateral direction of the first weight image.

In some embodiments, in response to the ultrasound waves being focused ultrasound beams, the first weight information is represented by a first weight image, the first weight image including one or more weighted regions, the first weight image is determined such that a width of the one or more weighted regions of the first weight image at a focal position of the focused ultrasound beams is narrower than that at other positions.

In some embodiments, in response to the ultrasound waves being focused ultrasound beams, the first weight information is represented by a first weight image, the first weight image is determined such that each of the plurality of composite sub-images includes an hourglass-type image region, or a combination of the plurality of composite sub-images is a complete image.

In some embodiments, the one or more weighted regions of the first weight image include a high-weighted region, and locations of high-weighted regions of different first weight images are different.

In some embodiments, the obtaining a plurality of original images produced based on a plurality of emissions of ultrasound waves includes: determining a stage scanning feature based on a stage scanning result of emitted ultrasound waves by using a scanning feature extraction layer of a parameter prediction model, the parameter prediction model being a machine learning model; and determining, by using a parameter prediction layer of the parameter prediction model, subsequent emission parameters and a count of subsequent emissions of the ultrasound waves based on, at least one of a historical count of emissions of emitted ultrasound waves, historical emission parameters of the emitted ultrasound waves, the stage scanning feature or a standard image.

In some embodiments, the method further comprising determining the first weight information based on at least one of a type of the ultrasound waves, a count of the plurality of weight data subsets, the plurality of original images or parameters corresponding to the plurality of original images.

In some embodiments, the method determining the first weight information using a first weight prediction model based on at least one of a type of the ultrasound waves, a count of the plurality of weight data subsets dataset emission parameters of the plurality of emissions of ultrasound waves, the plurality of original images, or parameters corresponding to the plurality of original images, the first weight prediction model being a machine learning model.

In some embodiments, the determining a target image based on a plurality of composite sub-images corresponding to the plurality of weight data subsets in the weight dataset includes: determining the target image based on the plurality of composite sub-images and second weight information, the second weight information being determined based on the plurality of composite sub-images using a second weight prediction model, the second weight prediction model being a machine learning model.

In some embodiments, the determining the second weight information further includes: dividing each composite sub-image into a plurality of sub-regions, and determining sub-image quality of each sub-region of the plurality of sub-regions, respectively, and determining sub-weight information of the each sub-region based on the sub-image quality; and determining the second weight information based on the sub-weight information.

In some embodiments, the second weight information includes third weight information and fourth weight information, and the second weight information is determined by: applying a low-pass filter or a speckle smoothing filter to the plurality of composite sub-images to generate an approximate image; generating a detailed image based on the plurality of composite sub-images and the approximate image; determining the third weight information and the fourth weight information using the second weight prediction model based on the approximate image and the detailed image, the third weight information being applied to the approximate image and the fourth weight information being applied to the detailed image.

One or more embodiments of the present disclosure provide a method for ultrasound imaging, implemented on a computing device having at least one processor and at least one storage device, the method comprising: obtaining a plurality of original images produced based on a plurality of emissions of ultrasound waves, emission angles, or emission positions corresponding to the plurality of emissions of ultrasound waves being different; for each of the plurality of original images, determining a plurality of weighted original images based on a plurality of first weight images; for each of the plurality of first weight images, determining a composite sub-image based on weighted original images corresponding to the first weight image; and determining a target image based on a plurality of composite sub-images corresponding to the plurality of first weight images.

One or more embodiments of the present disclosure provide a system for ultrasound imaging, comprising at least one storage device including a set of instructions, and at least one processor configured to communicate with the at least one storage device. When executing the set of instructions, the at least one processor is configured to direct the system to perform operations including: emitting a plurality of ultrasound waves, emission angles or emission positions corresponding to the plurality of emissions of ultrasound waves being different; obtaining a plurality of original images produced based on a plurality of emissions of ultrasound waves; for each original image of the plurality of original images, determining a weight dataset corresponding to the each original image, the weight dataset including first weight information, the first weight information including a plurality of pieces of first weight information, counts of the plurality of pieces of first weight information for the plurality of original images being the same; for each weight data subset in the weight dataset, determining a composite sub-image corresponding to the each weight data subset based on the plurality of original images and a plurality of pieces of first weight information corresponding to the each weight data subset; and determining a target image based on a plurality of composite sub-images corresponding to a plurality of weight data subsets in the weight dataset.

One or more embodiments of the present disclosure provide a non-transitory computer-readable medium, comprising at least one set of instructions, wherein when executed by at least one processor of a computer device, the at least one set of instructions directs the at least one processor to obtain a plurality of original images produced based on a plurality of emissions of ultrasound waves, emission angles, or emission positions corresponding to the plurality of emissions of ultrasound waves being different; for each original image of the plurality of original images, determine a weight dataset corresponding to the each original image, the weight dataset including a plurality of weight data subsets, a count of the plurality of weight data subsets for each of the plurality of original images being the same; for each weight data subset in the weight dataset, determine a composite sub-image corresponding to the each weight data subset based on the plurality of original images and a plurality of pieces of first weight information corresponding to the each weight data subset; and determine a target image based on a plurality of composite sub-images corresponding to the plurality of weight data subsets in the weight dataset.

Beneficial effects that may be brought about by some embodiments of the present specification include, but are not limited to: (1) using a plurality of pieces of first weight information can simulate the generation of a plurality of original images corresponding to different emission angles, thereby reducing an actual count of emissions of ultrasound waves at different emission angles, and thus reducing the effect of tissue movement; and through emission at different emission angles and emission positions, it is possible to reduce emission at a same emission angle and emission position. (2) a count of emission angles may be reduced, avoiding the reduction of spatial resolution and artifacts caused by tissue movement and the image trailing phenomenon due to tissue movement, thus reducing the speckle noise of a target ultrasound image and improving the quality of the target ultrasound image; (3) by designing first weight information of high-weighted regions of different widths and positions, a plurality of pieces of first weight information adapted to different ultrasound beams can be obtained, thus simulating ultrasound images corresponding to different emission angles. It should be noted that the beneficial effects that may be produced by different embodiments are different, and the beneficial effects that may be produced in different embodiments may be any one or a combination of the foregoing, or any other beneficial effect that may be obtained.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering denotes the same structure, where:

FIG. 1 is a schematic diagram illustrating an exemplary application scenario of a system for ultrasound imaging according to some embodiments of the present disclosure;

FIG. 2 is a schematic diagram illustrating exemplary modules of the system for ultrasound imaging according to some embodiments of the present disclosure;

FIG. 3 is a flowchart illustrating an exemplary method for ultrasound imaging according to some embodiments of the present disclosure;

FIG. 4A is a schematic diagram illustrating generation of a composite sub-image according to some embodiments of the present disclosure;

FIG. 4B is a schematic diagram illustrating generation of the composite sub-image according to some other embodiments of the present disclosure;

FIG. 5A is a schematic diagram illustrating first weight information of wide-beam ultrasound waves according to some embodiments of the present disclosure;

FIG. 5B is a schematic diagram illustrating first weight information of another wide-beam ultrasound waves according to some embodiments of the present disclosure;

FIG. 6 is a schematic diagram illustrating first weight information of focused ultrasound beams according to some embodiments of the present disclosure;

FIG. 7 is a schematic diagram illustrating a parameter prediction model according to some embodiments of the present disclosure; and

FIG. 8 is a schematic diagram illustrating a first weight prediction model according to some embodiments of the present disclosure;

FIG. 9 is a flowchart illustrating an exemplary method for determining a target image according to some embodiments of the present disclosure;

FIG. 10a is a schematic diagram illustrating determining a weighted original image according to some embodiments of the present disclosure;

FIG. 10b is a schematic diagram illustrating determining a composite sub-image according to some embodiments of the present disclosure; and

FIG. 10c is a schematic diagram illustrating determining a target image according to some embodiments of the present disclosure.

### DETAILED DESCRIPTION

In order to more clearly illustrate the technical solutions of the embodiments of the present disclosure, the accompanying drawings required to be used in the description of the embodiments are briefly described below. The accompanying drawings do not represent the entirety of the embodiments.

It should be understood that the terms “system”, “device”, “unit” and/or “module” as used herein is a way to distinguish between different components, elements, parts, sections or assemblies at different levels. The words may be replaced by other expressions if other words accomplish the same purpose.

Unless the context clearly suggests an exception, the words “a”, “an”, “one”, and/or “the” do not refer specifically to the singular, but may also include the plural. Generally, the terms “including” and “comprising” suggest only the inclusion of clearly identified steps and elements that does not constitute an exclusive list, and the method or apparatus may also include other steps or elements.

When describing the operations performed in the embodiments of the present disclosure in a step-by-step manner, the order of the steps is all interchangeable if not otherwise indicated, the steps can be omitted, and other steps can be included in the process of operation.

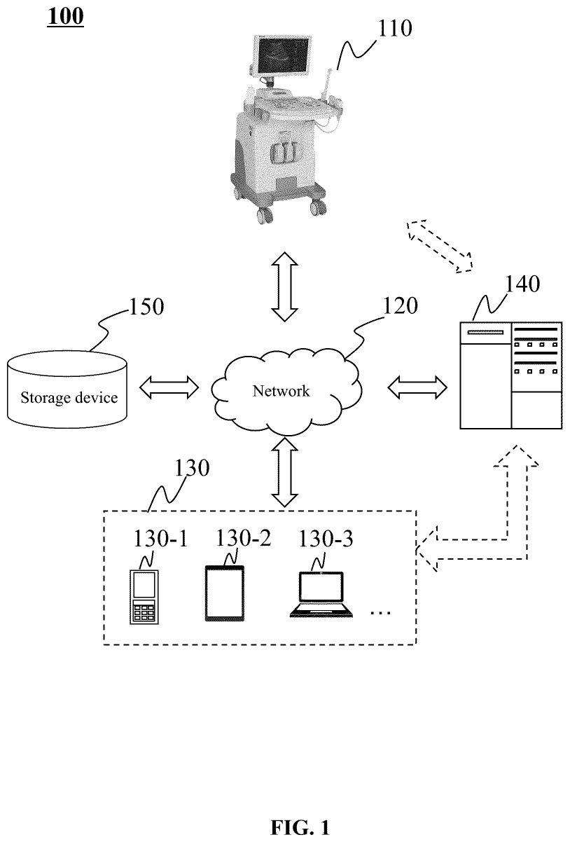

FIG. 1 is a schematic diagram illustrating an application scenario of a system for ultrasound imaging according to some embodiments of the present disclosure.

As shown in FIG. 1, an application scenario of a system 100 for ultrasound imaging may include an ultrasound imaging device 110, a network 120, a terminal 130, a processing device 140, and a storage device 150. Components in the system 100 may be connected in various ways. For example, the ultrasound imaging device 110 may be connected to the processing device 140 via the network 120, as shown in FIG. 1. The ultrasound imaging device 110 may be directly coupled to the processing device 140 (as indicated by a bi-directional arrow in dashed lines connecting the ultrasound imaging device 110 and the processing device 140). The storage device 150 may be connected to the processing device 140 directly or the processing device 140 through the network 120.

The ultrasound imaging device 110 may be used to acquire ultrasound data of a target object. For example, it may be used to acquire ultrasound data of an imaging region (e.g., organs and/or tissues such as the head, chest, abdomen, heart, blood vessels, etc.) of the target object (e.g., a patient). The ultrasound data may be presented in various forms such as waveforms, curves, or images. In some embodiments, the ultrasound imaging device 110 may emit different types of ultrasound beams (e.g., a broad beam, a focused ultrasound beam, etc.) at the imaging region or a portion of the imaging region of the target object, and receive and process ultrasound echo data reflected from the imaging region. In some embodiments, the ultrasound imaging device 110 may generate an image based on the ultrasound echo data to generate an ultrasound image for display. In some embodiments, the ultrasound imaging device 110 may send the ultrasound data and/or the ultrasound image to the processing device 140 via the network 120 to enable processing of the ultrasound data (e.g., composition of the ultrasound image(s)).

The network 120 may include any suitable network that facilitates the exchange of information and/or data for the system 100. In some embodiments, one or more components of the system 100 (e.g., the ultrasound imaging device 110, the terminal 130, the processing device 140, or the storage device 150) may transmit information and/or data with one or more other components of the system 100 via the network 120. For example, the processing device 140 may obtain ultrasound data of a scanned object from the ultrasound imaging device 110 via the network 120. In some embodiments, the network 120 may be any one or more of a wired network or a wireless network. In some embodiments, the network may be any of a variety of topologies, such as peer-to-peer, shared, centralized, or a combination of topologies.

The terminal 130 may include a mobile device 130-1, a tablet 130-2, a laptop 130-3, etc., or any combination thereof. In some embodiments, the terminal 130 may interact with other components in the system 100 via the network 120. For example, the terminal 130 may receive the ultrasound data sent by the ultrasound imaging device 110. In some embodiments, the terminal 130 may receive information and/or instruction entered by a user (e.g., a user of the ultrasound imaging device 110, such as a physician) and send the information and/or instruction to the ultrasound imaging device 110 or the processing device 140 via the network 120. For example, the physician may observe the quality of the ultrasound data (e.g., the ultrasound image) (e.g., speckle noise removal) through the terminal 130.

The processing device 140 may process data and/or information obtained from the ultrasound imaging device 110, the terminal 130, and/or the storage device 150. For example, the processing device 140 may acquire ultrasound data of a person being scanned. In some embodiments, the processing device 140 may process the ultrasound data. For example, the processor may perform a composition operation on a plurality of ultrasound images of a region of interest.

In some embodiments, the processing device 140 may be a single server or a group of servers. The group of servers may be centralized or distributed. In some embodiments, the processing device 140 may be local or remote. The processing device 140 may be directly connected to the ultrasound imaging device 110, the terminal 130, and the storage device 150 to access stored or acquired information and/or data. In some embodiments, the processing device 140 may be implemented on a cloud platform. Merely by way of example, the cloud platform may include a private cloud, a public cloud, a hybrid cloud, a community cloud, a distributed cloud, an on-premises cloud, a multi-tier cloud, etc., or any combination thereof.

The storage device 150 may store data and/or instructions. In some embodiments, the storage device 150 may store data obtained from the ultrasound imaging device 110, the terminal 130, and/or the processing device 140. For example, the storage device 150 may store ultrasound data obtained by a user after the user scans a device, etc. In some embodiments, the storage device 150 may store data and/or instructions used by the processing device 140 to perform exemplary methods described in the present disclosure. For example, the storage device 150 may store instructions for the processing device 140 to perform methods shown in flowcharts. In some embodiments, the storage device 150 may include a mass storage device, a removable storage device, volatile read-write memory, read-only memory (ROM), etc., or any combination thereof. In some embodiments, the storage device 150 may be implemented on a cloud platform. In some embodiments, the storage device 150 may be part of the processing device 140.

The above descriptions are for illustrative purposes only, and actual application scenarios may have various variations.

It should be noted that the system 100 is provided for illustrative purposes only and is not intended to limit the scope of the present disclosure. For a person of ordinary skill in the art, a variety of modifications or variations may be made in accordance with the description in the present disclosure. However, these changes and modifications do not depart from the scope of the present disclosure.

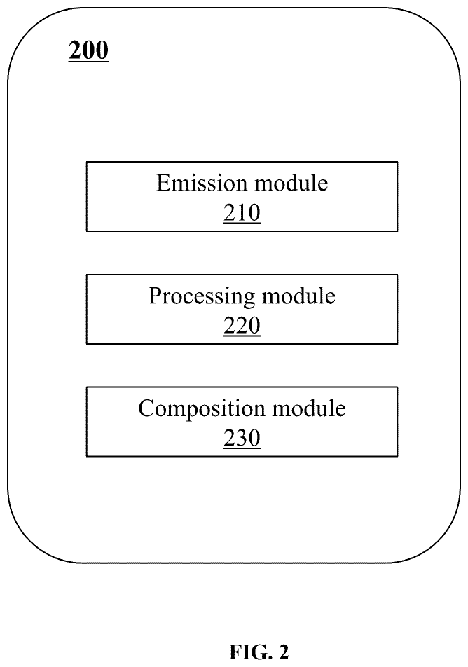

FIG. 2 is a schematic diagram illustrating exemplary modules of a system for ultrasound imaging according to some embodiments of the present disclosure.

In some embodiments, a system 200 for ultrasound imaging may include an emission module 210, a processing module 220, and a composition module 230, as shown in FIG. 2.

The emission module 210 may be configured to emit ultrasound waves for a plurality of times.

The processing module 220 may be configured to obtain a plurality of original images produced based on a plurality of emissions of ultrasound waves, and, for each original image of the plurality of original images, determine a weight dataset corresponding to the each original image. The processing module 220 may be configured to determine, for each weight data subset in the weight dataset, a composite sub-image corresponding to the each weight data subset based on the plurality of original images and first weight information corresponding to the each weight data subset.

The composition module 230 may be configured to determine a target image based on a plurality of composite sub-images corresponding to a plurality of weight data subsets in the weight dataset.

In some embodiments, in response to the ultrasound waves being wide beams, the first weight information may be determined such that each of the composite sub-images is a complete image or a combination of the plurality of composite sub-images is a complete image. In response to the ultrasound waves being focused ultrasound beams, the first weight information may be determined such that each of the plurality of composite sub-images includes an hourglass-type image region, or a combination of the plurality of composite sub-images a complete image. In response to the ultrasound waves being focused ultrasound beams, the first weight information may be represented by a first weight image, the first weight image including one or more weighted regions, the first weight image is determined such that a width of the one or more weighted regions of the first weight image at a focal position of the focused ultrasound beams is narrower than that at other positions. More relevant content can be found in the related description below.

In some embodiments, the processing module 220 may determine emission parameters and/or a count of the emissions of the ultrasound waves based on an imaging region of the ultrasound imaging and/or a feature of the imaging region.

In some embodiments, the processing module 220 may determine a stage scanning feature based on a stage scanning result of emitted ultrasound waves by using a scanning feature extraction layer of a parameter prediction model. The processing module 220 may determine, by using a parameter prediction layer of the parameter prediction model, subsequent emission parameters and a count of subsequent emissions of the ultrasound waves based on, at least one of a historical count of emissions of emitted ultrasound waves, historical emission parameters of the emitted ultrasound waves, the stage scanning feature or a standard image.

In some embodiments, the processing module 220 may determine a count of a plurality of weight data subsets based on a coverage range of the ultrasound waves.

In some embodiments, the processing module 220 may determine the count of a plurality of weight data subsets using a sequence length model based on the imaging region of the ultrasound imaging, the feature of the imaging region, and the coverage range of the ultrasound waves.

In some embodiments, the processing module 220 may determine the first weight information based on at least one of a type of ultrasound waves, a count of the plurality of weight data subsets, and the plurality of original images, or parameters corresponding to the plurality of original images.

In some embodiments, the processing module 220 may determine the first weight information using a first weight prediction model based on at least one of the type of ultrasound waves, the count of the plurality of weight data subsets, and emission parameters of the plurality of emissions of ultrasound waves, the plurality of original images, or the parameters corresponding to the plurality of original images.

In some embodiments, the processing module 220 may determine features of the original images based on the plurality of original images using an image feature prediction layer. The processing module 220 may determine the first weight information based on at least one of the type of ultrasound waves, the count of the plurality of weight data subsets, the emission parameters of the plurality of emissions of ultrasound waves, the plurality of original images, or the parameters corresponding to the plurality of original images using an information prediction layer.

In some embodiments, the composition module 230 may determine a target image based on the plurality of composite sub-images and the second weight information.

In some embodiments, the composition module 230 may determine the second weight information using a second weight prediction model based on the plurality of composite sub-images, the first weight information, and the emission parameters of the ultrasound waves.

In some embodiments, the composition module 230 may apply a low-pass filter or a speckle smoothing filter to the plurality of composite sub-images to generate an approximate image, generate a detailed image based on the plurality of composite sub-images and the approximate image, and determine third weight information and fourth weight information using the second weight prediction model based on the approximate image and the detailed image.

For more content about the emission module 210, the processing module 220, and the composition module 230, please refer to the descriptions below.

It is noted that the system 200 for ultrasound imaging and its modules may be used by a processing device (e.g., the processing device 140) to realize the functions of one or more embodiments described for each module after executing computer instructions (e.g., program code). In some embodiments, the processing device may include one or more sub-processing devices (e.g., a single-core processing device or a multi-core processing device). Just as an example, a processing device may contain a central processing unit (CPU), an application-specific integrated circuit (ASIC), an application-specific instruction processor (ASIP), a graphics processor (GPU), a physical processor (PPU), a digital signal processor (DSP), a field-programmable gate array (FPGA), a programmable logic circuit (PLD), a controller, a microcontroller unit, a reduced instruction Set Computer (RISC), microprocessor, etc. or any combination thereof. In some embodiments, the processing device may include a corresponding storage medium (e.g., memory) or be signal-connected to an external storage medium.

It should be noted that the above description of the system 200 for ultrasound imaging and the modules thereof is provided only for descriptive convenience, and does not limit the present disclosure to the scope of the cited embodiments. It should be understood that for a person skilled in the art, with an understanding of the principle of the system, it may be possible to arbitrarily combine modules or form subsystems that are connected to other modules without departing from this principle. For example, the individual modules may share a common storage module, and the individual modules may each have their own storage module.

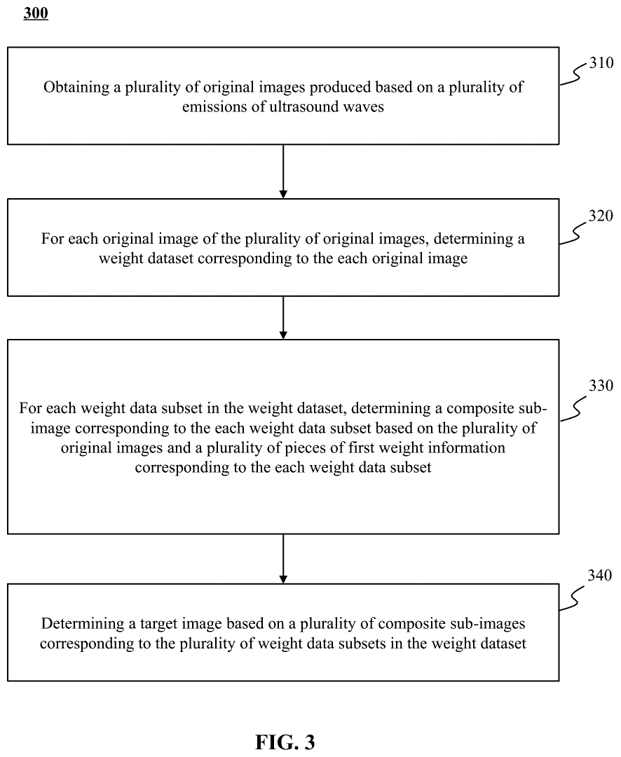

FIG. 3 is a flowchart illustrating an exemplary method for ultrasound imaging according to some embodiments of the present disclosure. In some embodiments, a process 300 may be implemented on a computer device having at least one processor and a storage device. As shown in FIG. 3, the process 300 may include one or more of the following operations.

In 310, a plurality of original images produced based on a plurality of emissions of ultrasound waves may be obtained.

An original image corresponds to data obtained by performing ultrasound scanning or ultrasound detection on an imaging region of a target object.

The target object may be an object for which the ultrasound scanning or the ultrasound detection is performed, which may be a living thing such as a human or an animal, or a part thereof. For example, the target object may be a patient for lesion detection or a pregnant woman for fetal morphology detection.

The imaging region is a region that needs to be scanned and analyzed by ultrasound imaging. For example, the imaging region may be an organ and/or tissue such as the head, chest, abdomen, heart, blood vessels, or the like. The imaging region includes features of a plurality of different imaging regions (e.g., a type, a location, a shape, an area, etc., of a tissue), and/or includes acoustic impedance and attenuation feature corresponding to different organs and/or tissues. The imaging region may be pre-set based on medical diagnostic needs. The feature of the imaging region may be obtained, e.g., by an image recognition algorithm.

The original image may be represented as an image or as a matrix. When represented as a matrix, elements in the matrix are represented as gray values of pixel points in two-dimensional coordinates. When represented as an image, the original image may include any feasible image such as a two-dimensional grayscale image, a three-dimensional grayscale image, and so on.

A system for ultrasound imaging may emit ultrasound waves (an incident ultrasound signal) into the imaging region or a portion thereof through an ultrasound probe of an ultrasound imaging device (e.g., the ultrasound imaging device 110) and receive reflected ultrasound waves (an ultrasound echo signal) from the imaging region. The ultrasound imaging device may process the ultrasound echo signal (e.g., acoustic-electric signal conversion, etc.) to obtain the original image.

In some embodiments, the system for ultrasound imaging may obtain one or more original images using the ultrasound imaging device based on one or more emissions of ultrasound waves. One original image may be obtained by emitting ultrasound waves one time.

The ultrasound probe of the ultrasound imaging device may include an ultrasound array including a plurality of (e.g., 128, 256, etc.) ultrasound array elements. Each ultrasound array element may be used to emit and/or receive an ultrasound signal. In some embodiments, each ultrasound array element may have an array element number, and the system for ultrasound imaging may control ultrasound array elements of a preset count to emit ultrasound waves for one or more times.

In some embodiments, emission angles or emission positions corresponding to the plurality of emissions of ultrasound waves being different. In some embodiments, a single emission of ultrasound waves may be defined by emission parameters. For example, the emission parameters may include information such as an emission time of the ultrasound waves, an emission position, an emission angle, a count of array elements, a coverage area, etc. The system for ultrasound imaging may determine the emission parameters (e.g., the emission angle, the count of array elements, etc.) based on the imaging region and the feature of the imaging region, and relevant information of the ultrasound imaging device. For example, for a line array probe, a plurality of original images may be obtained by emitting ultrasound waves to different positions. As another example, for a convex array probe, ultrasound waves may be emitted at different angles to obtain a plurality of original images, and the convex array probe may emit combined ultrasound waves at different angles and different positions to enable simulation of data emitted from other angles and reduce a count of emissions.

In some embodiments, if a plurality of ultrasound waves are emitted by a phased array, and the ultrasound imaging device uses a same aperture each time the ultrasound waves are emitted, then the emission angle may be varied by moving a focal point in accordance with an arc trajectory. The focal point is a position where the ultrasound waves are focused.

In some embodiments, if focal points of a plurality of emissions of ultrasound waves are distributed on a same plane, a portion of the ultrasound array elements among all ultrasound array elements are used in each emission of ultrasound waves, then the focal point of the emissions of ultrasound waves may translate by using different array elements in the each emission.

In some embodiments, if a depth of the imaging region is relatively deep, it may be necessary to increase a depth of the focal point by using all ultrasound array elements. At this time the movement of the focal point is controlled by varying an emission time of each array element, so as to change an angle of the focal point through emission delay.

In some embodiments, the plurality of emissions of ultrasound waves may be emitted at different emission positions and/or emission angles. That is, the plurality of original images are obtained by emitting ultrasound waves at different emission angles and/or different emission positions. The emission position refers to a position where the ultrasound probe is located. The emission angle refers to an angle between the ultrasound waves emitted by the ultrasound probe and a region of interest.

Exemplarily, the system for ultrasound imaging may control r (e.g., 30) adjacent first ultrasound array elements to simultaneously emit ultrasound waves at an emission angle (or direction) g₁ at a moment t₁ to accomplish emission of ultrasound waves one time, and control r (e.g., 30) adjacent second ultrasound array elements to simultaneously emit ultrasound waves at an emission angle g₂ at a moment t₂ to accomplish another emission of ultrasound waves. Similarly, the ultrasound imaging system emits ultrasound waves multiple times during a preset time period to achieve full coverage (area) of the imaging region. The ultrasound waves are emitted multiple times at different emission angles. As another example, the system for ultrasound imaging may control the 30 first ultrasound array elements to simultaneously emit ultrasound waves at an emission position p₁ at the moment t₁ to accomplish the emission of ultrasound waves one time; and at the moment t₂, control the 30 first ultrasound array elements to simultaneously emit ultrasound waves at an emission position p₂ to accomplish another emission of ultrasound waves. At this time, the plurality of emissions of ultrasound waves are emitted at different emission positions. It should be noted that emission angles and emission positions of the plurality of emission of ultrasound waves may be different, and ultrasound array elements in adjacent emissions of ultrasound waves may be completely different, partially the same, or the same.

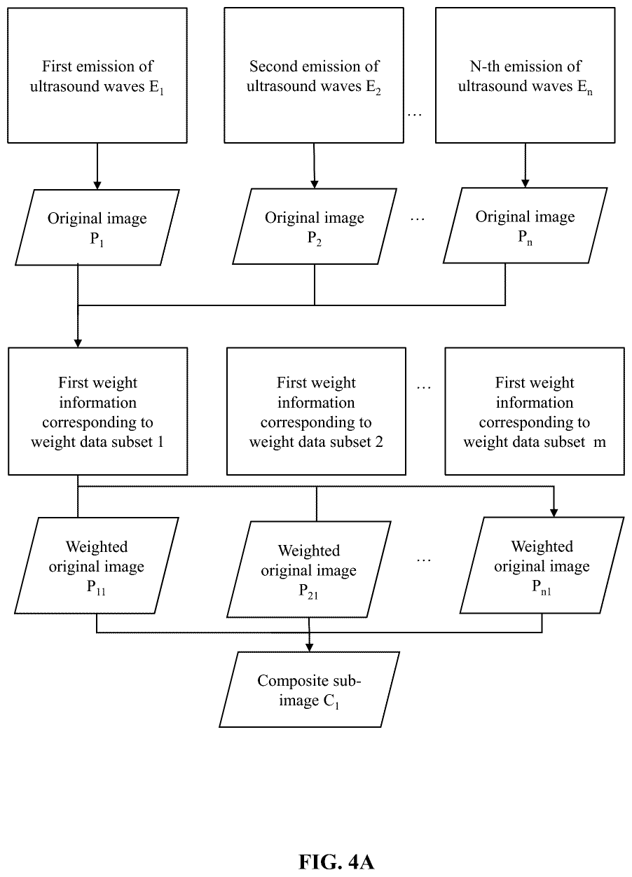

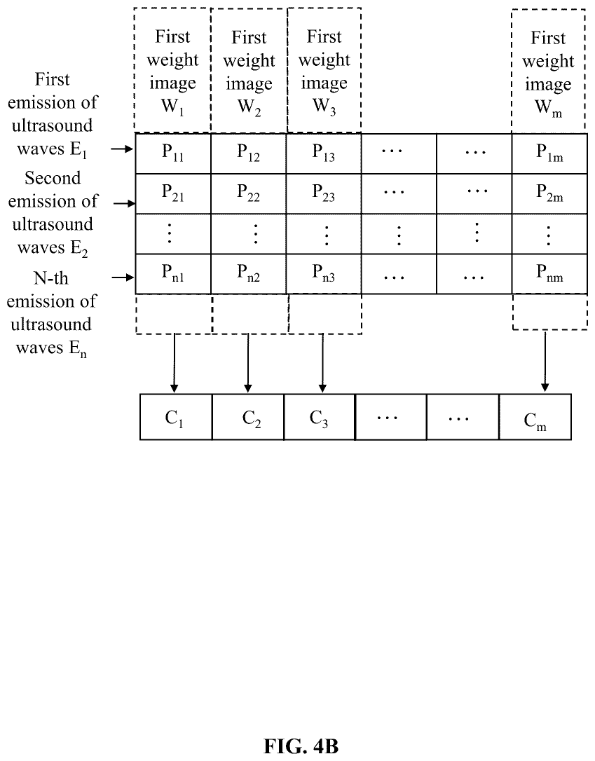

It will be appreciated that the system for ultrasound imaging may generate a single original image corresponding to the imaging region based on an ultrasound echo signal received from a single emission of ultrasound waves, and accordingly, a plurality of emissions of ultrasound waves may generate a plurality of original images. Each original image may be denoted by a weight data subset, e.g., I(i) denotes an original image, i=1, 2, . . . , n. FIG. 4A is a schematic diagram illustrating generation of a composite sub-image according to some embodiments of the present disclosure. As shown in FIG. 4A, emission of ultrasound waves for n (e.g., 10, etc.) times may generate n original images including an original image P₁, an original image P₂, . . . , an original image Pₙ.

In some embodiments, a system for ultrasound imaging may determine emission parameters and/or a count of emissions for a plurality of emissions of ultrasound waves in a variety of ways based on an imaging region of ultrasound imaging and a feature of the imaging region. For example, the system for ultrasound imaging may, based on the imaging region of the ultrasound imaging and the feature of the imaging region, query for a reference count of emissions and reference emission parameters for each emission corresponding to the imaging region and the feature of the imaging region in a parameter preset table. The reference count of emissions and the reference emission parameters for each emission are determined as a count of emissions of ultrasound waves in multiple emissions and emission parameters for each emission.

The parameter preset table may be pre-set based on historical data, including a plurality of groups of imaging regions and features of the imaging regions and a reference count of emissions and reference emission parameters for each emission corresponding to each group of imaging regions and the feature of the imaging regions. The reference emission parameters and the reference count of emissions may be determined based on manual labeling.

In some embodiments, the system for ultrasound imaging may determine, based on the imaging region of the ultrasound imaging and the feature of the imaging region, the emission parameters and/or the count of emissions for multiple emissions of ultrasound waves using a parameter prediction model.

The parameter prediction model refers to a model for determining the emission parameters and/or the count of emissions for multiple emissions of ultrasound waves. In some embodiments, the parameter prediction model may be a machine learning model. For example, the parameter prediction model may include any one or a combination of, for example, a Convolutional Neural Networks (CNN) model, a Neural Networks (NN) model, or other customized models.

In some embodiments, the system for ultrasound imaging may train and obtain the parameter prediction model by, for example, a gradient descent manner based on a large number of first training samples with first labels. Each group of training samples of the first training samples may include a sample imaging region and a sample feature of the sample imaging region. The first label of the first training sample may be an actual count of emissions and actual emission parameters for each emission. In some embodiments, the first training sample may be obtained based on historical data. The system for ultrasound imaging may select an actual count of emissions and actual emission parameters for each emission corresponding to a sample imaging region that meets a preset requirement in the historical data and as the first label. The preset requirement may include an amount of speckle noise of a target image corresponding to the sample imaging region being less than a noise threshold. The noise threshold may be pre-set based on historical experiences. More content about the target image can be found in operation 340 and its related descriptions.

In some embodiments, the parameter prediction model may be trained as follows: inputting a plurality of first training samples with a first label into an initial parameter prediction model, constructing a loss function from the first label and a prediction result of the initial parameter prediction model, updating the initial parameter prediction model iteratively based on the loss function, and a training of the parameter prediction model is completed when the loss function of the initial parameter prediction model satisfies a preset condition. The preset condition may be that the loss function converges, a count of iterations reaches a set value, or the like.

In some embodiments of the present disclosure, based on the imaging region of the ultrasound imaging and the features of the imaging region, more appropriate emission parameters and/or a count of emissions can be determined, thereby avoiding situations where too few or too many original images are produced due to an inappropriate count of emissions as well as original images not meeting medical diagnostic needs due to inappropriate emission parameters.

In some embodiments, the emission parameters may include subsequent emission parameters and a count of subsequent emissions for subsequent emissions of ultrasound waves. The subsequent emissions of ultrasound waves refer to ultrasound waves to be emitted after the plurality of emissions of ultrasound waves. Subsequent emission parameters are emission parameters for each emission in the subsequent emissions of ultrasound waves. The count of subsequent emissions refers to a count of times the ultrasound waves are emitted in subsequent emissions.

In some embodiments, the parameter prediction model may include a scanning feature extraction layer and a parameter prediction layer. The system for ultrasound imaging may determine a stage scanning feature based on a stage scanning result of emitted ultrasound waves by using the scanning feature extraction layer of the parameter prediction model, and determine the subsequent emission parameters and the count of subsequent emissions of the ultrasound waves by using the parameter prediction layer of the parameter prediction model based on at least one of a historical count of emissions of emitted ultrasound waves, historical emission parameters of emitted ultrasound waves, the stage scanning feature, or a standard image. More content about the above-described section can be found in FIG. 7 and its related descriptions thereof.

In 320, for each original image of the plurality of original images, a weight dataset corresponding to the each original image may be determined.

The weight dataset is a dataset including a plurality of weight data subsets. The weight dataset allows for performing a weighted processing on the original image. In some embodiments, the plurality of weight data subsets may correspond to a plurality of pieces of first weight information, and a count of a plurality of weight data subsets for each of a plurality of original images being the same.

In some embodiments, the weight dataset may be represented by a matrix or an image. For example, if represented by a matrix, elements of the matrix may represent weight information for corresponding pixel points in the original image at the time a target image is generated. If represented by an image, the weight dataset may be a grayscale image, and a brightness difference of the grayscale image may reflect a weight difference, and pixel points with larger weights have larger brightness. A description of the target image can be found in operation 340 and its associated description. In some embodiments, the weight dataset may also be in other forms, e.g., sequence, table, text, or the like.

In some embodiments, the weight dataset may be in form of a weight sequence. The weight sequence is a sequence including first weight information. The original image may be weighted by the weight sequence. In some embodiments, the weight sequence may include a plurality of pieces of first weight information, with counts of the plurality of pieces of first weight information for the plurality of original images being the same. In some embodiments, the first weight information may be represented by a matrix or image. If represented by a matrix, elements in the matrix may represent weight information of corresponding pixel points in the original image when generating a target image. If represented by an image, the first weight information may be a grayscale image, and a difference in brightness of the grayscale image may reflect a difference in weight, with pixel points of greater weight having greater brightness. A description of the target image can be found in operation 340 and its related description.

One piece of first weight information may correspond to one piece of weight information of a pixel point in the original image at the time of generating the target image. In some embodiments, one original image may correspond to one weight dataset since a weight dataset may include weight information of different pixel points of the original image at the time of generating the target image.

Exemplarily, the weight dataset corresponding to the each original image may be W(i, j), where i in W(i, j) denotes a weight data subset of the original image, j denotes a j-th piece of first weight information in the weight dataset corresponding to the original image i, j=1, 2, . . . , m, m denotes a length of the weight dataset, i=1, 2, . . . , n, n denotes a count of the original image.

In some embodiments, at least two pieces of first weight information corresponding to a same weight data subset among at least two of the plurality of weight datasets are the same. For example, a weight dataset corresponding to a first original image may be W(1, j), a weight dataset corresponding to a second original image may be W(2, j), then first weight information of W(1, 1) and W(2, 1) may be the same, and first weight information of W(1, 2) and W(2, 2) may be the same.

In some embodiments, if there are at least two pieces of first weight information corresponding to a same being the same among the plurality of the weight datasets corresponding to the plurality of original images, this allows for similar weighted processing on the plurality of original images, and thus obtaining a more homogeneous composite sub-image.

In some embodiments, the first weight information may include a weight value corresponding to each position in the plurality of original images. The weight value may represent weight information of the each position in the original image when generating the target image. The position in the original image may include positions where pixel points or voxel points are located. For example, each position may be a location where each pixel point or voxel point in the original image is located.

In some embodiments, due to different emission angles, etc., resulting in a different region being scanned each time, positions of a same imaging region are different under different original images, so weight values of the same imaging region under different original images may be different, which in turn contribute differently to a generated target image, and a target image that better reflects the imaging region may be generated. A description of the target image can be found in operation 340 and its associated description.

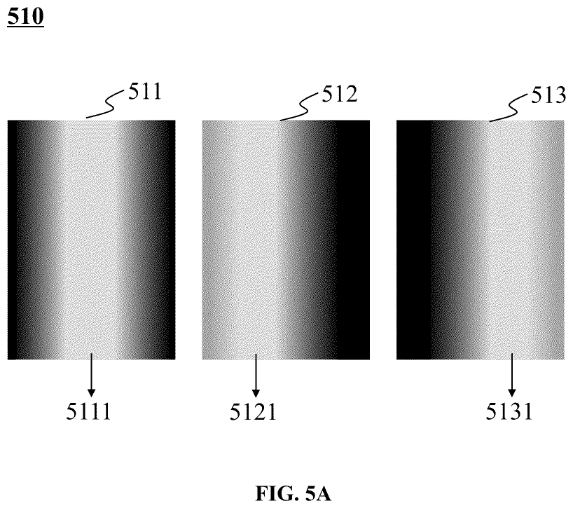

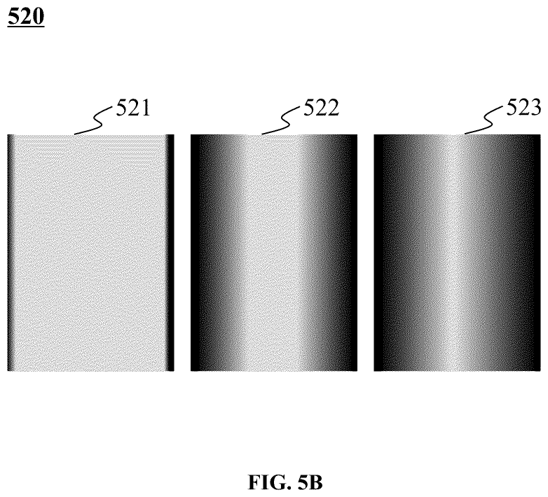

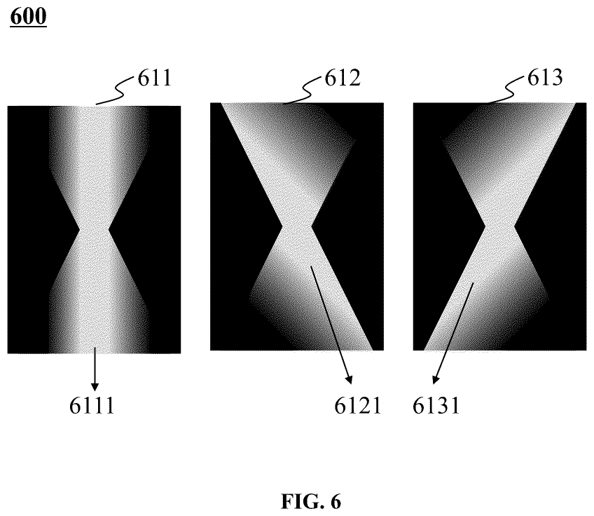

In some embodiments, the first weight information may be represented in a form of a first weight image (e.g., a grayscale image, etc.). The first weight image is as shown in FIG. 5A, FIG. 5B, and FIG. 6. A brightness value of a pixel point in the weight image is positively correlated with a weight value corresponding to the pixel point, and a higher brightness indicates a higher weight value. In some embodiments, the weight information may be represented in a form of a weight matrix corresponding to the weight image, with a value of each element in the matrix representing a weight value.

In some embodiments, a preset emission angle may correspond to a count of emissions, with one preset emission angle corresponding to one emission of ultrasound waves, and a plurality of different preset emission angles corresponding to a plurality of emissions of ultrasound waves.

The first weight information may be determined according to actual situations. In some embodiments, the system for ultrasound imaging may determine a count of pieces of first weight information based on a feature of the imaging region and ultrasound emission information (e.g., emission parameters, etc.). For example, a plurality of pieces of first weight information may be set based on a plurality of different preset emission angles for a plurality of emissions of ultrasound waves. The plurality of different preset emission angles may be determined based on an area of the imaging region and a coverage range of the imaging region by each emission of ultrasound waves. If the coverage range is relatively large, the more preset emission angles (e.g., 10°, 20°, 45°, etc.) may be used, and the more the count of pieces of first weight information is. It should be noted that the first weight information may be pre-configured or determined based on an actual situation of a plurality of emissions of ultrasound waves (e.g., a brightness distribution of regions in the original image, etc.).

In some embodiments, the system for ultrasound imaging may determine the first weight information in a variety of ways based on at least one of a type of ultrasound waves, the count of a plurality of weight data subsets, the plurality of original images, or parameters corresponding to the plurality of original images. For example, the system for ultrasound imaging may determine initial first weight information corresponding to the type of ultrasound waves based on the type of ultrasound waves via a preset weight table. The system for ultrasound imaging may obtain a plurality of pieces of extended first weight information by performing operations such as translating, rotating, or the like on each piece of initial first weight information, and, based on the each piece of initial first weight information and the plurality of pieces of extended first weight information, generate a plurality of groups of weight information. The system for ultrasound imaging may determine, based on the plurality of groups of weight information and the plurality of original images, target images corresponding to the groups of weight information in operations 330 and 340, or the like, and determine a group of weight information corresponding to a target image with the best imaging effect as the first weight information. The system for ultrasound imaging may combine one piece of initial first weight information and any count of pieces of extended first weight information among the plurality of pieces of extended first weight information to form one group of weight information. The system for ultrasound imaging may obtain the plurality of pieces of extended first weight information by performing operations such as translating, rotating, or the like on the each piece of initial first weight information using a window function. A description of the window function can be found in a related description below. A description of the target image can be found in operation 340 and its related description.

Parameters corresponding to the original image may include a variety of, for example, at least one of an emission focus, an emission aperture, and an emission spacing, etc. corresponding to emission of ultrasound waves when the original image is obtained. The parameters corresponding to the original image may be obtained by an ultrasound imaging device.

The type of ultrasound waves may include at least one of wide beams, plane beams, focused ultrasound beams, or the like. The group of weight information may include one piece of initial first weight information and any count of pieces of extended first weight information. A count of the extended first weight information may be the count of a plurality of o weight data subsets.

The preset weight table may be pre-set to include a plurality of types of ultrasound waves and initial first weight information corresponding to each type of ultrasound waves. The initial first weight information may be determined by manual labeling.

In some embodiments, the imaging effect of the target image corresponding to the group of weight information may be determined using an effect prediction model.

The effect prediction model is a model for determining the imaging effect of the target image. In some embodiments, the effect prediction model may be a machine learning model. For example, the effect prediction model may include any one or a combination of a Graph Neural Network (GNN) model, a Neural Networks (NN) model, or other customized model structures, etc.

In some embodiments, the system for ultrasound imaging may train and obtain the effect prediction model in a gradient descent manner, for example, based on a large number of second training samples with a second label. Each set of training samples of the second training sample may include a plurality of sample original images and a sample group of weight information. The second label of the second training sample may be imaging effect of a target image corresponding to the plurality of sample original images. In some embodiments, the second training sample may be obtained based on historical data. The imaging effect of the target image corresponding to the plurality of sample original images may be expressed by values in a range (e.g., 0-1), with a larger value indicating a better imaging effect.

In some embodiments, the second label may be determined based on a manual assessment of an amount and distribution of speckle noise of the target image corresponding to the plurality of sample original images. For example, the greater the amount and the wider the distribution of the speckle noise of the target image, the worse the imaging effect.

In some embodiments, a training process of the effect prediction model is similar to a training process of the parameter prediction model, and an implementation manner thereof can be referred to an implementation manner of the training process of the parameter prediction model.

In some embodiments of the present disclosure, based on the type of ultrasound waves, first weight information suitable for different types of ultrasound waves may be determined, and the imaging effect of the target image can be obtained quickly using a machine learning model, which is conducive to determining more accurate first weight information.

In some embodiments, the system for ultrasound imaging may determine the first weight information based on the type of ultrasound waves, the length of the weight dataset, and the emission parameters of the plurality of emissions of ultrasound waves, the plurality of original images, or parameters corresponding to the plurality of original images using a first weight prediction model. The system for ultrasound imaging may input emission parameters for one emission of ultrasound waves at a time, and output, through the first weight prediction model, first weight information corresponding to emission parameters of that one emission, and through inputting emission parameters multiple times, first weight information corresponding to emission parameters for multiple emissions of ultrasound waves may be obtained.

The first weight prediction model is a model for determining the first weight information. In some embodiments, the first weight prediction model may be a machine learning model. In some embodiments, the first weight prediction model may include any one or a combination of, for example, a Convolutional Neural Networks (CNN) model or any other customized models.

In some embodiments, the system for ultrasound imaging may train and obtain the first weight prediction model by a gradient descent manner, for example, based on a large number of third training samples with a third label. Each set of training samples of the third training sample may include at least one of a type of sample ultrasound waves, a count of a plurality of sample weight data subsets, and sample emission parameters for each emission of ultrasound waves, a plurality of sample original images, or parameters corresponding to the plurality of sample original images. The third label may include standard first weight information corresponding to the type of sample ultrasound waves. In some embodiments, the third training sample may be determined based on historical data.

In some embodiments, for each type of ultrasound waves, the system for ultrasound imaging may generate the group of weight information in a manner described in operation 420 and, based on the target image corresponding to the group of weight information, a group of weight information corresponding a target image that has the best imaging effect is determined as the standard first weight information, and standard first weight information corresponding to the each type of ultrasound waves is determined as the third label.

In some embodiments, a training process of the first weight prediction model is similar to the training process of the parameter prediction model, and an implementation manner thereof may be referred to an implementation manner of the training process of the parameter prediction model.

In some embodiments of the present disclosure, by using the first weight prediction model, first weight information that can make the imaging effect of the target image better can be quickly obtained based on a large amount of complex data and a clarified correlation relationship between input content and an output result, which is favorable to generate a target image that is more suitable for medical diagnosis.

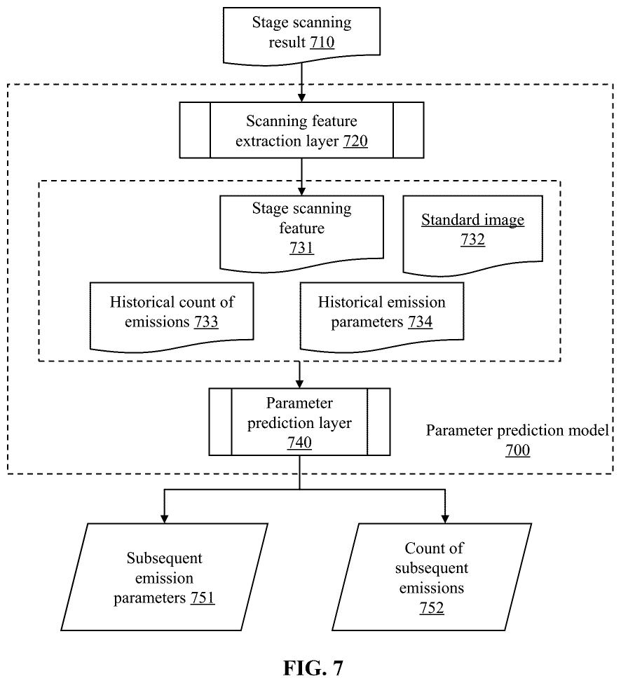

In some embodiments, the first weight prediction model may include a scanning feature extraction layer and a parameter prediction layer. The system for ultrasound imaging may determine, based on a plurality of original images, features of the original images determined using an image feature prediction layer, the type of ultrasound waves, the count of the plurality of weight data subsets, the emission parameters for the plurality of emissions of ultrasound waves, and the features of the original images, the first weight information using the information prediction layer. More content about this part can be found in FIG. 8 and its related description.

In some embodiments, the first weight information represented in a form of a weight image (e.g., a grayscale image, etc.) may be a first weight image. Each first weight image includes one or more weighted regions, and different first weighted regions include different weighted regions. A dimension of the first weight image is the same as a dimension of the original image.

The weighted region is a region whose weight value is not 0 in the first weight image. For example, a pixel value of a pixel point within the weighted region may be set to be a value within (0, 255], which is characterized as a region with a certain brightness in the first weight image. It is noted that in some embodiments, the system for ultrasound imaging may obtain a weight value corresponding to each pixel based on pixel values in the first weight image after mapping to a specific range of values (e.g., [0, 1]) for a weighted processing.

The unweighted region is a region whose weight value is 0 in the first weight image. For example, a pixel value of a pixel point within the unweighted region is set to 0, which is characterized as a black region in the first weight image.

In some embodiments, the system for ultrasound imaging may construct a plurality of first weight images corresponding to a same original image and having different weighted regions. For example, weighted regions with different areas, contours, and shapes may be constructed. An area of weighted regions and a spatial distribution of the weighted regions may also be different for different first weight images.

More content about the first weight image can be found in FIGS. 5A, 5B, 6 and their descriptions.

In 330, for each weight data subset in the weight dataset, a composite sub-image corresponding to the each weight data subset may be determined based on the plurality of original images and a plurality of pieces of first weight information corresponding to the each weight data subset.

For example, a weight dataset corresponding to each original image may be W(i, j), where i in W(i, j) denotes a weight data subset of the original image, j denotes an j-th piece of first weight information in a weight dataset corresponding to the original image i, j=1, 2, . . . , m, m denotes a count of a plurality of the weight dataset i=1, 2, . . . , n, n denotes a count of original images. If a composite sub-image corresponding to a weight data subset 1 is determined, a plurality of pieces of first weight information corresponding to the weight data subset 1 are W(1, 1), W(2, 1), . . . , W(n, 1).

The composite sub-image is an ultrasound image generated by performing coherent compounding on a plurality of weighted original images. In some embodiments, for a plurality of weighted original images corresponding to a plurality of pieces of first weight information corresponding to each weight data subset, the system for ultrasound imaging may also process the plurality of weighted original images, such as arranging or stitching, to generate the composite sub-image.

In some embodiments, for each weight data subset in the weight dataset, the system for ultrasound imaging may obtain a plurality of weighted original images of a plurality of pieces of first weight information corresponding to the weight data subset, and generate a composite sub-image. In some embodiments, a plurality of emissions of ultrasound waves at different emission angles may achieve full coverage of an imaging region, and thus each composite sub-image may correspond to a complete ultrasound image of an imaging region corresponding to a certain piece of weight information.

The weighted original image is an ultrasound image obtained after the original image is subjected to a weighted processing based on the first weight information. In some embodiments, for an original image, the system for ultrasound imaging may generate a weighted original image by performing the weighted processing on the original image based on one piece of first weight information in a weight dataset corresponding to the original image. For example, the one piece of first weight information in the weight dataset corresponding to the original image may be multiplied with the original image to obtain the weighted original image. A plurality of weighted original images may be generated by performing the weighted processing on the original image based on each piece of first weight information in the weight dataset corresponding to the original image.

A process of performing the weighted processing on the original image based on the first weight information may be viewed as a process of assigning importance to pixels in the original image, and the larger the weight value corresponding to a pixel, the greater the importance assigned to the pixel, and the greater its influence on a subsequently-generated target image.

In some embodiments, each original image is generated utilizing a plurality of ultrasound signals emitted from a plurality of array elements, with the plurality of ultrasound signals having different emission angles. The first weight information may be used to assign different importance to echoes generated by different paths of ultrasound signals. For example, a particular piece of weight information may be configured to assign greater importance to echoes generated by an ultrasound signal in a 10-degree direction, and thus image data corresponding to the ultrasound signal in a 10-degree in the weighted original image has greater brightness. By setting different first weight information, different importance may be assigned to image data corresponding to ultrasound signals at different angles. Using the plurality of pieces of first weight information may simulate the generation of a plurality of original images corresponding to different emission angles, thereby reducing an actual count of emissions of ultrasound waves emitted at different emission angles, and thus reducing the effect of tissue movement.

FIG. 4A is a schematic diagram illustrating generation of a composite sub-image according to some embodiments of the present disclosure.

FIG. 4B is a schematic diagram illustrating generation of a composite sub-image according to some other embodiments of the present disclosure.

As shown in FIG. 4A, for an original image P₁ generated by a first emission of ultrasound waves E₁, an original image P₂ generated by a second emission of ultrasound waves E₂, . . . , an original image Pₙ generated by an n-th emission of ultrasound waves Eₙ, a system for ultrasound imaging may utilize first weight information corresponding to a weight data subset 1 to perform a weighted processing on the original image P₁, P₂, . . . , and Pₙ, to generate a weighted original image P₁₁, a weighted original image P₂₁, . . . , a weighted original image Pₙ₁, and the system for ultrasound imaging may perform a composition processing on the weighted original image P₁₁, the weighted original image P₂₁, . . . , the weighted original image Pₙ₁ to generate a composite sub-image C1. Similarly, for a plurality of emissions of ultrasound waves, the system for ultrasound imaging may generate a composite sub-image corresponding to each weight data subset. The first weight information corresponding to a weight data subset 1 refers to first weight information whose weight data subset is 1 among all weight datasets.

As shown in FIG. 4B, the imaging system may obtain a plurality of weighted original images {P₁₁, P₁₂, P₁₃ . . . . P₁ₘ} generated by the first emission of ultrasound waves E₁. The plurality of weighted original images {P₁₁, P₁₂, P₁₃, . . . , P₁ₘ} may be generated by a first weight image W₁, a first weight image W₂, a first weight image W₃, . . . , a first weight image Wm performing a weighted processing on an ultrasound sub-image generated by the first emission of ultrasound waves E₁, respectively. More content about determining the weighted original image can be found in the related description above.

Similarly, the system for ultrasound imaging may obtain a plurality of weighted original images {P₂₁, P₂₂, P₂₃, . . . , P₂ₘ}, . . . , {Pₙ₁, Pₙ₂, Pₙ₃, . . . , Pₙₘ} generated by the second emission of ultrasound waves E₂, . . . , the n-th emission of ultrasound waves Eₙ, respectively.

The system for ultrasound imaging may perform the weighed processing on a weighted original image (i.e., a plurality of weighted ultrasound sub-images in a same column in FIG. 4B) corresponding to each first weight image to obtain a composite sub-image corresponding to the each first weight image. For example, as shown in FIG. 4B, the system for ultrasound imaging may coherently combine weighted ultrasound sub-images {P₁₁, P₁₂, . . . , P₁ₘ} to obtain a composite sub-image C₁ corresponding to the first weight image W₁. Therefore, the system for ultrasound imaging may obtain a composite sub-image C₁, a composite sub-image C₂, a composite sub-image C₃, . . . , a composite sub-image Cm corresponding to the first weight image W₁, the first weight image W₂, the first weight image W₃, . . . , the first weight image Wm.

In some embodiments of the present disclosure, by setting a plurality of pieces of different first weight information to perform the weighted processing on the original image, it is possible to obtain a plurality of weighted original images of a same emission of ultrasound waves, and spatially composite the plurality of weighted original images to obtain a composite sub-image. The conventional solution requires setting a plurality of emission angles and/or a plurality of reception angles for each emission. Compared with the conventional solution, the embodiments in the present disclosure can reduce the emission angle and/or the reception angle, avoiding the spatial resolution degradation and the image trailing phenomenon caused by the tissue movement, thereby reducing the speckle noise of a target image that improves the quality of the target image.

In 340, the target image may be determined based on a plurality of composite sub-images corresponding to a plurality of weight data subsets in the weight dataset.

The target image is an ultrasound image corresponding to a complete imaging region that is ultimately generated, which can be used to be presented on a display device for a user (e.g., a physician, an operator, etc.) to observe and analyze an imaging region of a target object. The display device may include a device that displays the target image to the user. The user may include a physician, etc.

The target image may be in a form of a two-dimensional image, for example, an image of a slice of a heart organ at a certain moment of the target object, etc. The target image may still be in a form of other things, such as generating a three-dimensional image of an organ or tissue based on the reconstruction of a two-dimensional image under a plurality of different viewpoints, and so on.

In some embodiments, the system for ultrasound imaging may perform a spatial composition processing on a plurality of composite sub-images of a plurality of emissions of ultrasound waves to generate the target image. The spatial composition processing may be realized using techniques such as ultrasound spatial composition imaging (Compound Imaging).

In some embodiments, the system for ultrasound imaging may determine the target image in multiple ways based on the plurality of composite sub-images and a plurality of pieces of second weight information corresponding to a plurality of weight data subsets in a weight dataset. For example, for each composite sub-image, the system for ultrasound imaging may perform a signal processing on the plurality of composite sub-images to obtain a plurality of post-processing composite sub-images, perform a weighted processing (e.g., multiplying) on the plurality of post-processing composite sub-images based on the second weight information to obtain a plurality of weighted composite sub-images, and perform summing or averaging on the plurality of weighted composite sub-images to obtain the target image.

In some embodiments, the system for ultrasound imaging may perform the signal processing on the plurality of composite sub-images using various signal processing techniques.

In some embodiments, the system for ultrasound imaging may perform an envelope detection processing on the composite sub-image using envelope detection techniques including, but not limited to, amplitude modulation detection, moving average detection, or the like.

The envelope detection processing may be used to turn a high-frequency oscillation signal in the composite sub-image into a low-frequency signal to reduce speckle noise in the composite sub-image. In some embodiments, the envelope detection processing may be implemented using Hilbert transform algorithm.

The post-processing composite sub-image refers to an ultrasound image of the composite sub-image after the envelope detection processing. For example, the system for ultrasound imaging may perform the envelope detection processing on each of the composite sub-images, respectively, to generate a corresponding post-processing composite sub-image.

The second weight information may be used to perform the weighted processing on the post-processing composite sub-images. In some embodiments, the second weight information may be in a form of a second weight image (e.g., a grayscale image, etc.). In some embodiments, the system for ultrasound imaging may determine the second weight image based on a quality analysis of the composite sub-image or a post-processing composite sub-image corresponding to the composite sub-image.

The quality analysis may be used to assess imaging effect of the composite sub-images. In some embodiments, the system for ultrasound imaging may perform the quality analysis on the composite sub-images and/or the post-processing composite sub-images using various types of image analysis algorithms. For example, the quality analysis may be an assessment of a brightness uniformity, a clarity, or the like, of the composite sub-images and/or the post-processing composite sub-images.

In some embodiments, a result of the quality analysis may be determined based on an assessment value, with a larger assessment value indicating a better imaging effect. The assessment value may be a combination of one or more factors including, but not limited to, a signal-to-noise ratio, contrast (e.g., differentiation of a region of interest from surrounding tissues), resolution, brightness uniformity, or the like, of the composite sub-images (e.g., the composite sub-images, the post-processing composite sub-images). Exemplarily, a signal-to-noise ratio of the composite sub-image may be determined based on an assessment of a speckle noise situation of the composite sub-image, with a larger signal-to-noise ratio, a larger assessment value, or a brightness uniformity of the composite sub-image may be assessed, with a larger uniformity resulting in a larger assessment value. In some embodiments, the composite sub-image may be assessed for quality by regions to obtain assessment values of different sub-regions.

In some embodiments, weight values of different regions in the second weighted region may be determined based on assessment values of different regions in the composite sub-images and/or the post-processing composite sub-images. For example, if an assessment value of a region in the composite sub-image and/or the post-processing composite sub-image is below a threshold value, a weight value of each pixel within a second weighted region corresponding to the region may be lower, otherwise the weight value may be larger. In this way, image information of regions with higher assessment values may be retained more in a final generated target image, while the influence of regions with smaller assessment values on the final generated target image can be attenuated to improve the quality of the target image (e.g., improving the brightness uniformity and signal-to-noise ratio).

In some embodiments, each point in the second weight information has a same weight value, and the weight value may be determined based on an assessment value of an entire composite sub-image. At this point, the second weight information may be considered as a numerical value or a matrix, a weight image (the second weight image), etc.

In some embodiments, the system for ultrasound imaging may directly perform summing and averaging on the plurality of post-processing composite sub-images to generate the target image. For example, the post-processing composite sub-image may first be summed to obtain a summed image. For each pixel point in the summed image, a pixel value of the each pixel point may be determined as a sum of corresponding pixel values of how many post-processing composite sub-images. Assuming that a pixel value of a certain pixel point is a sum of pixel values of N post-processing composite sub-mages, the pixel value of the pixel point may be divided by N as a pixel value of a corresponding point in the target image.

In some embodiments of the present disclosure, by performing the envelope detection processing on the composite sub-images, it is possible to reduce the speckle noise of each composite sub-image before compositing to obtain the target image, which in turn provides a good base of image quality for the generation of the target image; furthermore, by performing the quality analysis on the composite sub-images and/or the post-processing composite sub-images, more targeted second weight information may be obtained, which improves the quality of the target image.

In some embodiments, the system for ultrasound imaging may determine image quality of the plurality of composite sub-images using, for example, a machine learning model or a preset algorithm, and, based on the image quality, determine the second weight information using a preset relationship. The machine learning model may include any feasible model such as MF R-CNN, L-CNN, and C-CNN. The preset algorithm may include any feasible algorithm such as FR-IQA, RR-IQA, and NR-IQA. The preset relationship may include that the better the image quality of the composite sub-image, the larger the weight value of the second weight information corresponding to the composite sub-image.

In some embodiments of the present disclosure, a weight value corresponding to a composite sub-image with better image quality may be set larger to obtain a target image with a better imaging effect.

In some embodiments, the system for ultrasound imaging may divide each composite sub-image into a plurality of sub-regions according to a same specification, determine sub-image quality of each sub-region, respectively, determine sub-weight information based on the sub-image quality, and determine the second weight information based on the sub-weight information. Each of the plurality of sub-regions into which the composite sub-image is divided has a same count, a same position, a same size, etc. A specification of image division refers to a size of the sub-region, which may be pre-set based on historical experience, for example, an image may be divided into nine equal parts, and a shape of each piece of the image may be a square, etc.

In some embodiments, the system for ultrasound imaging may divide the composite sub-image in a variety of ways. For example, the composite sub-image is divided based on pixels or voxels, with one or more pixels or voxels being a sub-region.

In some embodiments, the system for ultrasound imaging may divide a composite sub-image into nine equal parts, each of which is a square and each of which is a sub-region. The system for ultrasound imaging may determine sub-image quality of each sub-region using, for example, the above-described machine learning model or a preset algorithm.

In some embodiments, the system for ultrasound imaging may determine, based on sub-image quality of sub-regions at a same position in each composite sub-image, sub-weight information corresponding to sub-regions at that position in each composite sub-image through a preset sub-relationship. The preset sub-relationship may include the better the sub-image quality, the greater the weight value of the sub-weight information corresponding to the sub-image quality.

Exemplarily, if there are three composite sub-images, selecting a sub-region with a highest sub-image quality of sub-regions at a same position among the three composite sub-images, setting sub-weight information of the sub-region to a maximum value among three pieces of sub-weight information; selecting a sub-region with the lowest sub-image quality of the sub-regions at a same position among the three composite sub-images, setting sub-weight information of the sub-region to a minimum value among the three pieces of sub-weight information, and selecting a sub-region with a middle sub-image quality and set sub-weight information of the sub-region to a middle value among the three pieces of sub-weight information.

In some embodiments, when one or more pixel points or voxel points are a sub-region, the system for ultrasound imaging may determine sub-weight information of the one or more pixel points or voxel points based on sub-image quality of the one or more pixel points or voxel points and a surrounding pixel point or voxel point. The surrounding pixel point or voxel point may be a pixel point or voxel point adjacent to the one or more pixel points or voxel points. For example, in the case of the pixel point, the system for ultrasound imaging may calculate an average of sub-image qualities of the pixel point and pixel points surrounding the pixel point, and based on the average, determine sub-weight information corresponding to the pixel point through the preset sub-relationship.

In some embodiments, the system for ultrasound imaging may form the second weight information based on all pieces of sub-weight information corresponding to all sub-regions of the composite sub-image.

In some embodiments of the present disclosure, the composite sub-image may be divided into a plurality of regions, and by comparing image qualities of same regions separately and determining corresponding weight information based on the image qualities, a more targeted weight information for different regions of the composite sub-image may be obtained.

In some embodiments, the system for ultrasound imaging may perform a weighted processing on composite sub-regions at the same position of each composite sub-image based on the sub-regions at the same position of each composite sub-image and a corresponding sub-weight of each sub-region and obtain a corresponding sub-region in the target image. A same operation is performed on a sub-region at each position of each composite sub-image to obtain the target image stitched together from a plurality of sub-regions.

In some embodiments, the system for ultrasound imaging may determine the second weight information based on a plurality of composite sub-images corresponding to a plurality of weight data subsets in the weight dataset using a second weight prediction model.

The second weight prediction model is a model for determining the second weight information. In some embodiments, the second weight prediction model may be a machine learning model. For example, the second weight prediction model may include any one or a combination of, for example, a Convolutional Neural Networks (CNN) model or other customized models.

In some embodiments, the system for ultrasound imaging may train and obtain the second weight prediction model based on a large number of fourth training samples with fourth labels in a gradient descent manner. Each set of training samples of the fourth training samples may include a plurality of sample composite sub-images, and the fourth label may include standard second weight information corresponding to the sample composite sub-image. In some embodiments, the fourth training sample may be determined based on historical data. The plurality of sample composite sub-images may be obtained based on a plurality of sample original images corresponding to the sample composite sub-images in the historical data in operation 330, etc.

In some embodiments, the system for ultrasound imaging may determine a plurality of different pieces of second weight information corresponding to the plurality of sample composite sub-images in the above-described manner for determining the second weight information. The system for ultrasound imaging may generate sample target images based on the plurality of sample composite sub-images and second weight information corresponding to each of the plurality of sample composite sub-images, and determine second weight information corresponding to a sample target image with the best imaging effect as the standard second weight information. The ultrasound imaging system may determine imaging effect of the sample target image using quality analysis or the like. A process of determining the imaging effect of the sample target image is similar to the above-described process of determining the imaging effect of the composite sub-image, and can be found in the above-described implementation manner of the process of determining the imaging effect of the composite sub-image.

In some embodiments, a training process of the second weight prediction model is similar to a training process of the parameter prediction model, and an implementation manner of the second weight prediction model can be referred to an implementation manner of the training process of the parameter prediction model.

In some embodiments of the present disclosure, based on the composite sub-image, the second weight prediction model allows for quick determination of more reasonable second weight information, which is conducive to obtaining a target image more suitable for medical diagnosis.

In some embodiments, the second weight information may include third weight information and fourth weight information. The system for ultrasound imaging may apply a low-pass filter or a speckle smoothing filter to the composite sub-images to generate an approximate image; generate a detailed image based on the composite sub-images and the approximate image, and determine the third weight information and the fourth weight information based on the approximate image and the detailed image using the second weight prediction model. The third weight information refers to data used for a weighted processing on the approximate image, and the third weight information may include weight information corresponding to each pixel point or voxel point in the approximate image, and the fourth weight information refers to data used for a weighted processing on the detailed image, and the fourth weight information may include weight information corresponding to each pixel point or voxel point in the detailed image. The low-pass filter or the speckle smoothing filter is a device used to filter a specific frequency.

In some embodiments, the system for ultrasound imaging may determine filter parameters of the low-pass filter or the speckle smoothing filter in a variety of ways based on a type of filter, the first weight information corresponding to the composite sub-image, and the imaging region. For example, the system for ultrasound imaging may query, based on the type of filter, the first weight information corresponding to the composite sub-image, and the imaging region, a preset parameter table for reference filter parameters corresponding to the type of filter, the first weight information corresponding to the composite sub-image, and the imaging region, and determine the reference filter parameters as filter parameters of the low-pass filter or filter parameters of the speckle smoothing filter. The type of filter may be obtained by user input, etc.

The filter parameters are parameters related to an operation of a filter. In some embodiments, the filter parameters may include at least one of a passband, a stopband, a cutoff frequency, a passband gain, a stopband attenuation, or the like. The preset parameter table may be pre-set based on historical data, including a plurality of groups of types of filters, first weighting information, and imaging regions, as well as reference filter parameters corresponding to each group.

In some embodiments, the reference filter parameters may be determined through multiple experiments. For example, for a group of composite sub-images that have a same type of filter, first weight information, and imaging region, one of the composite sub-images is selected as an experimental sub-image. The system for ultrasound imaging may randomly change one or more parameters of preset filter parameters to generate a plurality of different experimental filter parameters based on the preset filter parameters, and apply the low-pass filter or the speckle smoothing filter to the experimental sub-image based on the plurality of different experimental filter parameters to generate a plurality of groups of experimental approximate images and experimental detailed images. The system for ultrasound imaging may generate a plurality of groups of approximate images and detailed images by applying the low-pass filter or the speckle smoothing filter to composite sub-images in a group of composite sub-images other than the experimental sub-images based on the preset filter parameters. The system for ultrasound imaging may perform the weighted processing on the plurality of groups of approximate images, the plurality of detailed images, one group of experimental approximate images, and one group of experimental detailed images to obtain an experimental target image based on preset third weight information and preset fourth weight information. A plurality of experimental target images may be obtained by repeating a process of generating the experimental target image, experimental filter parameters corresponding to an experimental target image with the best imaging effect may be used as reference filter parameters corresponding to the group of composite sub-images, that is, the reference filter parameters corresponding to a type of filter, first weight information, and imaging region corresponding to the group of composite sub-images. The preset filter parameters are parameters related to the operation of the filter, and may be set in advance based on historical experience. The preset third weight information refers to data set in advance for the weighted processing on the approximate image. The preset fourth weight information is data set in advance for the weighted processing on the detailed image. The preset third weight information and the preset fourth weight information may be set in advance based on historical experience. A process of determining imaging effect of the experimental target image is similar to a process of determining the imaging effect of the target image, and can be referred to the process of determining the imaging effect of the target image.

In some embodiments, the system for ultrasound imaging may determine the filter parameters based on the type of filter, the first weight information corresponding to the composite sub-image, and the imaging region using a filter parameter model.

The filter parameter model is a model used to determine the filter parameters. In some embodiments, the filter parameter model may be a machine learning model. For example, the filter parameter model may include any one or a combination of a Convolutional Neural Networks (CNN) model, a Neural Networks (NN) model, or other customized models, etc.

In some embodiments, the system for ultrasound imaging may train and obtain the filter parameter model based on a large number of seventh training samples with seventh labels in a gradient descent manner. Each set of training samples of the seventh training sample may include a type of sample filter, sample first weight information corresponding to a sample composite sub-image, and a sample imaging region, and the seventh label of the seventh training sample may be reference filter parameters corresponding to the seventh training sample. In some embodiments, the seventh training sample may be obtained based on historical data. The reference filter parameters may be determined by manual labeling, for example, a technician may evaluate the type of sample filter, the sample first weight information corresponding to the sample composite sub-image, and the sample imaging region based on historical experience and determine the reference filter parameters.

In some embodiments, a training process of the first weight prediction model is similar to the training process of the parameter prediction model, and an implementation manner thereof may be referred to an implementation manner of realizing the training process of the parameter prediction model.

In some embodiments of the present disclosure, based on the type of filter, the first weight information corresponding to the composite sub-image, and the imaging region, more appropriate filter parameters can be determined, which can in turn result in a more accurate approximate image, thereby obtaining a more accurate detailed image.

The approximate image is an image that contains a same low spatial frequency or structure as the composite sub-image. The detailed image is an image the approximate image has been removed from the composite sub-image. The low spatial frequency may include clutter, grating flaps, noise artifacts, etc., in a low-echo region of the composite sub-image.

In some embodiments, the system for ultrasound imaging may apply the low-pass filter or the speckle smoothing filter to the composite sub-image to generate the approximate image. The system for ultrasound imaging may remove the low spatial frequency or structure of the approximate image (i.e., the approximate image) from the composite sub-image to obtain the detailed image containing only a high spatial frequency or only speckle noise. The high spatial frequency may include the speckle noise, a high-resolution target, etc. For each composite sub-image, an approximate image and a detailed image corresponding to each composite sub-image may be obtained by the aforementioned manner of removing the approximate image.

In some embodiments, the system for ultrasound imaging may input approximate images and detailed images corresponding to all composite sub-images into the second weight prediction model to obtain the third weight information corresponding to the approximate image and the fourth weight information corresponding to the detailed image. When the inputs to the second weight prediction model include the approximate images and the detailed images corresponding to all composite sub-images, each set of training samples of the fourth training sample also includes a sample approximate image and a sample detailed image, and the fourth label may include third weight information and fourth weight information corresponding to the sample approximate image and the sample detailed image, respectively.

In some embodiments, the sample approximate image and the sample detailed image may be obtained by processing the composite sub-image in the historical data as a process of obtaining the approximate image and the detailed image as described above. The third weight information and the fourth weight information corresponding to the sample approximate image and the sample detailed image, respectively, may be determined by manual labeling. For example, a technician may determine, based on historical experience, the third weight information and the fourth weight information corresponding to the sample approximate image and the sample detailed image, respectively.

In some embodiments, the system for ultrasound imaging may perform the weighted processing (e.g., multiply) on a plurality of approximate images and a plurality of detailed images based on the third weight information and the fourth weight information, respectively, to obtain a plurality of weighted composite sub-images, and sum or average the plurality of weighted composite sub-images to obtain the target image.

In some embodiments of the present disclosure, by generating the approximate image and the detailed image, it is possible to analyze and process speckle noise without having to deal with noise such as clutter with low spatial frequency and to obtain more targeted second weighting information, thereby determining a more accurate target image.

In some embodiments of the present disclosure, a plurality of original images may be formed based on a plurality of emissions of ultrasound waves, and a plurality of weighted original images may be generated in conjunction with the first weight information in the weight dataset based on the original images, which can remove the speckle noise, etc., in the original image, then a plurality of weighted original images may be combined to obtain composite sub-images, and the composite sub-images may be combined to obtain a target image with a higher resolution and signal-to-noise ratio.

In some embodiments, the system for ultrasound imaging may perform the process 300 multiple times to obtain a target image sequence of a target object, for example, an image sequence reflecting hemodynamic changes in a cardiac vessel of the target object. The target image sequence can help users (e.g., doctors, etc.) make more comprehensive medical diagnoses.

It should be noted that the foregoing description of the process 300 is intended to be merely exemplary and illustrative, and does not limit the scope of application of the present disclosure. For those skilled in the art, various corrections and changes to the process calibration can be made under the guidance of the present disclosure. However, these corrections and changes remain within the scope of the present disclosure.

In some embodiments, in response to the ultrasound waves being wide beams, the first weight information is determined such that each of the plurality of composite sub-images is a complete image, or a combination of the plurality of composite sub-images is a complete image. The complete image is an ultrasound image that reflects a complete imaging region. The wide beams are a type of ultrasound waves.

FIG. 5A is a schematic diagram illustrating a first weight image of wide beams according to some embodiments of the present disclosure.

More content about the first weight image, a weighted region of the first weight image, and the composite sub-image can be found in FIG. 3, FIG. 4, and the related descriptions thereof.

As shown in FIG. 5A, for example only, first weight images for wide beams include a first weight image 511, a first weight image 512, and a first weight image 513.

In some embodiments, a weighted region of each of the first weight images includes a high-weighted region, and high-weighted regions in different first weight images have different positions or widths along a lateral direction of the first weight image.

As shown in FIG. 5A, each of the first weight image 511, the first weight image 512, and the first weight image 513 include an unweighted black region and a weighted non-black region, respectively. Among the weighted regions, the high-weighted region is a region with a relatively high brightness (a pixel value/a grayscale value). High-weighted regions in the first weight image 511, the first weight image 512, and the first weight image 513 have different locations and widths along the lateral direction of the first weight image. The lateral direction refers to a horizontal direction of the first weight image. For the first weight image 511, a high-weighted region is a vertical bar-like region located in a middle position along a lateral direction (such as a region 5111 in the first weight image 511). For the first weight image 512, a high-weighted region is a vertical bar-like region (such as a region 5121 in the first weight image 512) that is located to the left along the lateral direction and has a width that is less than a width of the high-weighted region in the first weight image 511. For the first weight image 513, a high-weighted region is a vertical bar-like region (such as a region 5131 in the first weight image 513) located in a rightward position along the lateral direction and has a width that is less than the width of the high-weighted region in the first weight image 511 while is the same as the width of the high-weighted region in the first weight image 512.

In some embodiments, the system for ultrasound imaging may adjust a lateral position and/or width of the high-weighted regions in the first weight image 511, the first weight image 512, and/or the first weight image 513 for different needs.

By setting high-weighted regions/weighted regions at different positions along the lateral direction, different importance may be assigned to beams at different emission angles, thus realizing the simulation of ultrasound images at different emission angles. For example, suppose that an ultrasound transducer makes one emission at an angle perpendicular (considered as emission at 0 degrees) to an imaging region, and an original image is obtained. When performing a weighted processing on the original image to generate a weighted original image, the first weight image 511 may be used to assign a greater importance to a beam at 0 degrees, the first weight image 512 may be used to assign a greater importance to a beam to the left of the beam at 0 degrees, and the first weight image 513 may be used to assign a greater importance to a beam to the right of the beam at 0 degrees. By using the first weight image 511, the first weight image 512, and the first weight image 513, it is possible to simulate ultrasound images at different emission angles based on the original image obtained by one emission.

In some embodiments, high-weighted regions in at least two first weight images may be obtained by applying a different window function at a same position in each of the first weight images, respectively. Different window functions may be used to obtain high-weighted regions with different parameters such as shape, width, height, area, or the like.

In some embodiments, the window function may include but is not limited to, a Hamming Window function, a Blackman Window function, a Rectangle Window function, or the like.

FIG. 5B is a schematic diagram illustrating a first weight image of another wide-beam ultrasound waves according to some embodiments of the present disclosure.

As shown in FIG. 5B, first weight images include a first weight image 521, a first weight image 522, and a first weight image 523. High-weighted regions in the first weight image 521, the first weight image 522, and the first weight image 523 are obtained by applying different window functions at a same position (e.g., a middle position). As shown in FIG. 5B, the high-weighted regions in the first weight image 521, the first weight image 522, and the first weight image 523 are located in a middle position and have different widths; regions on left and right sides of the high-weighted regions are unweighted regions (black regions on both sides).

In some embodiments of the present disclosure, a more targeted first weight image can be obtained by designing the high-weighted regions in different first weight images to have different positions or widths along the lateral direction of the images.

FIG. 6 is a schematic diagram illustrating a first weight image of focused ultrasound beams according to some embodiments of the present disclosure.

In some embodiments, ultrasound waves corresponding to each emission of ultrasound waves are focused ultrasound beams, and weighted regions in first weight images are designed such that each of a plurality of composite sub-images includes an hourglass-type image region, or a combination of the plurality of composite sub-images is a complete image. The hourglass-type image region refers to a region that is wide in upper and lower portions and narrow in a central portion. For example, the hourglass-type image region may be similar to a weighted region shown in FIG. 6, which includes two identical trapezoids that overlap on short sides of the first weight image to form a narrower portion at a central portion of the hourglass-type image region. The focused ultrasound beams are a type of ultrasound waves.

In some embodiments, for focused ultrasound beams whose focus is within an ultrasound image, an acoustic field imaging region as a whole appears to be funnel-shaped since the ultrasound beams converge at a focal position. In this scenario, a width of the weighted regions of the first weight image at the focal position of the focused ultrasound beams is narrower than that at other positions.

As shown in FIG. 6, for illustrative purposes only, first weight images include a first weight image 611, a first weight image 612, and a first weight image 613. Taking the first weight image 611 as an example, a center point of the image corresponds to a focus position of the focused ultrasound beams, and a weighted region is an hourglass-type region, with an upper part designed as an inverted trapezoid (or trapezoid-like shape) and a lower part designed as an orthogonal trapezoid (or trapezoid-like shape). The narrowest portion of the hourglass-type region is located at the center point of the image (i.e., the focus position) of the first weight image 611.

In some embodiments, when the original image is subjected to the weighted processing (e.g., multiplied) by the first weight image 611 to generate a weighted original image, the weighted original image has no image information (pixel values of pixel points are 0) in a region corresponding to an unweighted region in the first weight image 611, and a region corresponding to a weighted region contains the image information, and thus, an effective region of a weighted ultrasound sub-image is also presented as a region containing the hourglass-type image as in the first weight image 611.

Further, the system for ultrasound imaging may generate the composite sub-image based on the plurality of weighted original images, such that a composite sub-image corresponding to the first weight image 611 includes the hourglass-type image region, or a sum of composite sub-images corresponding to the first weight image 611 includes the hourglass-type image region.

In some embodiments of the present disclosure, a weighted ultrasound sub-image adapted for ultrasound waves being focused ultrasound beams may be obtained by designing the width of the weighted region in each first weight image to be narrower at the focal position of the focused ultrasound beams than at other positions.

In some embodiments, a weighted image of each first weight image of ultrasound waves being focused ultrasound beams includes a high-weighted region, and positions of high-weighted regions in different first weight images are different. In some embodiments, different positions may include different tilt angles. The tilt angle is an angle of inclination of the high-weighted region with respect to the lateral direction of the image.

As shown in FIG. 6, the first weight image 612 and the first weight image 613 may be designed based on a same principle as that of the first weight image 611, or the first weight image 612 and the first weight image 613 may be obtained by transformation based on the first weight image 611. The high-weighted region may be a region with a higher brightness in the hourglass-type region. In the first weight image 611, a high-weighted region is perpendicular to a lateral direction of the image (such as a region 6111 in the first weight image 611); in the first weight image 612, a high-weighted region is skewed counterclockwise with respect to a direction perpendicular to the lateral direction of the image (such as a region 6121 in the first weight image 612), and in the first weight image 613, a high-weighted region is skewed clockwise with respect to the direction perpendicular to the lateral direction of the image (e.g., a region 6131 in the first weight FIG. 613).

In some embodiments, the system for ultrasound imaging may obtain the first weight image 612 and the first weight image 613 by rotating the high-weighted region in the first weight image 611 around a center point of the image (i.e., the focal position). Alternatively, the system for ultrasound imaging may apply a triangular window function to the weighted region in the first weight image 611 to obtain the first weight image 612 and the first weight image 613.

In some embodiments of the present disclosure, by designing different first weight images having high-weighted regions with different tilt angles, it is possible to obtain a plurality of first weight images adapted to an application scenario of the focused ultrasound beams, thereby simulating ultrasound images corresponding to different emission angles.

FIG. 7 is a schematic diagram illustrating a parameter prediction model according to some embodiments of the present disclosure.

In some embodiments, a parameter prediction model 700 may include a scanning feature extraction layer 720 and a parameter prediction layer 740. In some embodiments, the scanning feature extraction layer 720 may be any one or a combination of, for example, a Convolutional Neural Networks (CNN) model or other customized models. The parameter prediction layer 740 may be any one or a combination of Neural Networks (NN) models or other customized models, etc. Further description of the parameter prediction model can be found in FIG. 3 and its related descriptions.

In some embodiments, an input into the scanning feature extraction layer 720 may include a stage scanning result 710 of emitted ultrasound waves, and an output of the scanning feature extraction layer 720 may include a stage scanning feature 731. Inputs to the parameter prediction layer may include at least one of the stage scanning feature 731, a standard image 732, a historical count of emissions of emitted ultrasound waves 733, or historical emission parameters of emitted ultrasound waves 734, and outputs of the parameter prediction layer 740 may include subsequent emission parameters 751 and a count of subsequent emissions 752. The standard image is a standardized image of an imaging region, e.g., the heart. The standard image may be generated preset.

Further description of the imaging region, the subsequent emission parameters, and the count of subsequent emissions can be found in FIG. 3 and its related description.

The emitted ultrasound waves are ultrasound waves that have already been emitted to the imaging region.

The historical count of emissions of emitted ultrasound waves 733 is a count of times the emitted ultrasound waves have been emitted. The historical emission parameters of emitted ultrasound waves 734 are emission parameters of each emission of emitted ultrasound waves. In some embodiments, a system for ultrasound imaging may automatically count the count of times the ultrasound waves have been emitted and record emission parameters of each emission of ultrasound waves.

The stage scanning result 710 is a target image corresponding to the emitted ultrasound waves. In some embodiments, the system for ultrasound imaging may acquire the target image corresponding to the emitted ultrasound waves by a method for ultrasound imaging described in FIG. 3, and weight information (e.g., first weight information, second weight information, etc.) used in a process of acquiring the target image corresponding to the emitted ultrasound waves may be pre-set based on historical experience.

The stage scanning feature 731 is used to reflect a feature of the stage scanning result. For example, scanning effect of various regions in the stage scanning result, imaging regions and features of the imaging regions, and feature information of a target image corresponding to emitted ultrasound waves, e.g., a resolution, a penetration rate, etc.

In some embodiments, the scanning feature extraction layer 720 and the parameter prediction layer 740 of the parameter prediction model 700 may be obtained by a joint training. The joint training may be performed in a gradient descent manner. When the scanning feature extraction layer and the parameter prediction layer are jointly trained, each set of training samples of a first training sample may include a sample stage scanning result, a sample historical count of emissions, sample historical emission parameters, and a first label may include an actual count of subsequent emissions and actually subsequent emission parameters. Further description of the first training sample, the first label can be found in FIG. 3 and its related descriptions.

In some embodiments, the system for ultrasound imaging may, based on the first training sample, subsequently perform a plurality of groups of emissions of ultrasound waves, and determine a count of emissions and emission parameters of each emission of ultrasound waves in a group of emissions corresponding to a target image with a best imaging effect as the first label. A count of emissions and emission parameters for each emission of ultrasound waves in the plurality of groups of emissions of ultrasound waves are different. The system for ultrasound imaging may determine a count of emissions and emission parameters of each emission of ultrasound waves for each group of emission based on the above-described manner of determining the count of emissions and the emission parameters.

In some embodiments, a joint training of the scanning feature extraction layer 720 and the parameter prediction layer 740 may include: inputting sample stage scanning results from a plurality of first training samples into an initial scanning feature extraction layer to obtain sample stage scanning features, inputting the sample stage scanning features and sample historical count of emission and sample historical emission parameters in the first training samples into an initial parameter prediction layer, constructing a loss function based on the first label and an output of the initial parameter prediction layer, synchronously updating the initial parameter prediction layer and the initial scanning feature extraction layer based on an iteration of the loss function, and when the initial parameter prediction layer and the initial scanning feature extraction layer satisfy a preset condition, a training of the parameter prediction model is completed. The preset condition may be that the loss function converges, a count of iterations reaches a set value, and so on.

In some embodiments of the present disclosure, utilizing the self-learning capability of the machine learning model can improve the accuracy and efficiency of determining the subsequent emission parameters and the count of subsequent emissions, and by jointly training the parameter prediction layer and the scanning feature extraction layer, the accuracy of determining the subsequent emission parameters and the count of subsequent emissions can be improved. By determining the count of subsequent emissions and emissions parameters of subsequent emissions of ultrasound waves based on a count of emissions and emission parameters of emitted ultrasound waves, it is possible to obtain a more reasonable count of original images, and thereby obtain a target image with a better imaging effect.

FIG. 8 is a schematic illustrating a first weight prediction model according to some embodiments of the present disclosure.

In some embodiments, inputs to a first weight prediction model 800 may include a plurality of original images 810. The first weight prediction model 800 may also include an image feature extraction layer 820 and an information prediction layer 840. A system for ultrasound imaging may determine a feature of an original image 831 based on the plurality of original images 810 using the image feature prediction layer 820, and determine first weight information 850 based on a type of ultrasound waves 321, a count of a plurality of a weight dataset 833, emission parameters of a plurality of emissions of ultrasound waves 834, and the feature of an original image 831 using the information prediction layer 840.

The feature of an original image may characterize an amount and distribution of speckle noise, etc. A description of the original image, the type of ultrasound waves, the count of a plurality of the weight dataset, the emission parameters of the plurality of emissions of ultrasound waves, and the first weight information can be found in FIG. 1 and its related description.

In some embodiments, the image feature extraction layer 820 and the information prediction layer 840 of the first weight prediction model 800 may be obtained by a joint training. The joint training may be performed in a gradient descent manner. Each set of training samples of a third training sample may include a plurality of sample original images, a type of sample ultrasound waves, a count of a plurality of a plurality of sample weight data subsets, and sample emission parameters. A third label of the third training sample may include standard first weight information corresponding to the type of sample ultrasound waves. Further description of the third training sample, the third label, and the standard first weight information can be found in operation 320 and its related description.

In some embodiments, the joint training of the image feature extraction layer 820 and the information prediction layer 840 may include: inputting a plurality of sample original images in a plurality of third training samples into an initial image feature extraction layer, obtaining features of the sample original images, inputting the features of the sample original images and types of sample ultrasound waves, lengths of sample weight datasets, and sample emission parameters in the third training samples into an initial information prediction layer, constructing a loss function based on the third label and an output of the initial information prediction layer, and synchronize updating the initial information prediction layer and the initial image feature extraction layer based on an iteration of the loss function, and when the initial information prediction layer and the initial image feature extraction layer satisfy a preset condition, a training of the first weight prediction model is completed. The preset condition may be that the loss function converges, a count of iterations reaches a set value, or the like.

In some embodiments, the input to the information prediction layer 840 may also include high-weighted region information 835. The high-weighted region information is information associated with a high-weighted region. For example, a high-weighted region information corresponding to a first weight image may be a rectangular image with specific regions boxed out, etc. If first weight information is in a form of a matrix, high-weighted region information corresponding to the first weight information may be a matrix in which a high-weighted region takes a value of 1 and the other regions take a value of 0, etc.

In some embodiments, if the input to the information prediction layer includes the high-weighted region information, the first weight information output by the first weight prediction model may include the high-weighted region. If the input to the information prediction layer includes the high-weighted region information, the third training sample may include sample high-weighted region information.

In some embodiments of the present disclosure, the accuracy and efficiency of weight values of a determined high-weighted region can be improved by presetting the high-weighted regions in the first weight information in advance and predicting weight values of the high-weighted regions using the first weight prediction model.

In some embodiments of the present disclosure, by jointly training the image feature extraction layer and the information prediction layer, it is beneficial to solve problems such as the difficulty of training the image feature extraction layer and the information prediction layer individually, and it is beneficial to further improve the accuracy of determined first weight information, which in turn facilitates the subsequent determination of a composite sub-image.

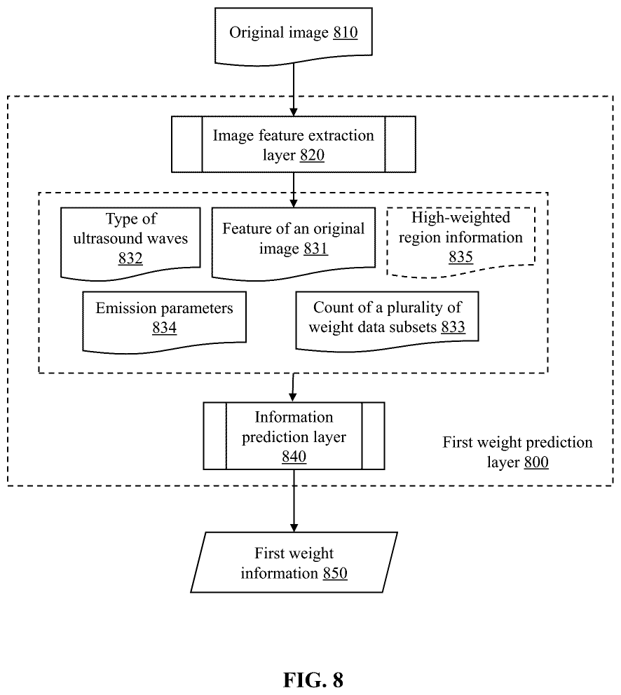

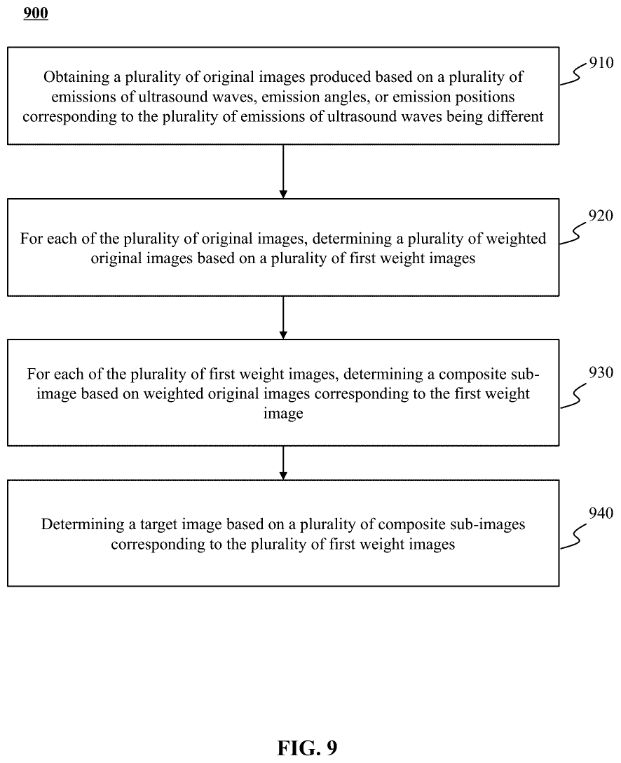

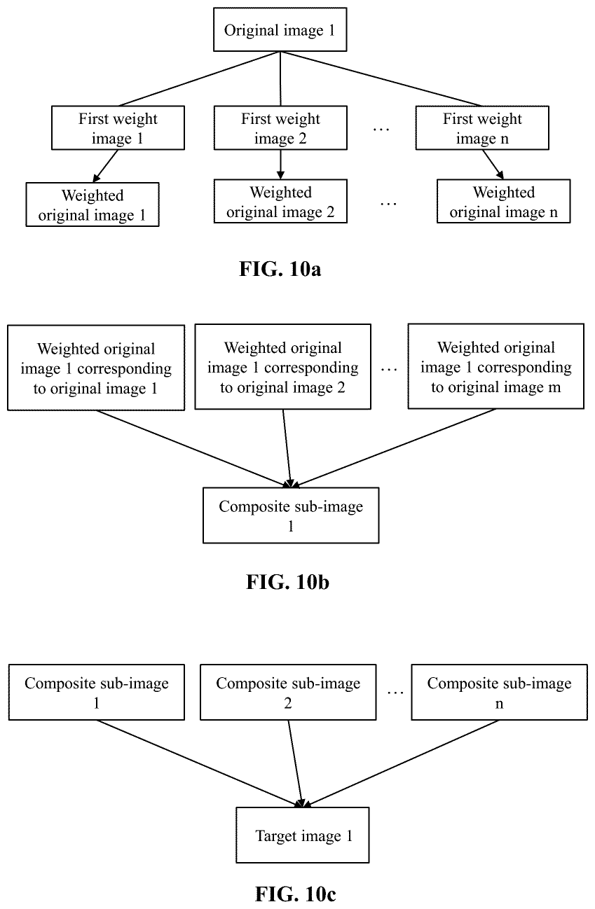

FIG. 9 is a flowchart illustrating an exemplary method for determining a target image according to some embodiments of the present disclosure. FIG. 10a is a schematic diagram illustrating determining a weighted original image according to some embodiments of the present disclosure; FIG. 10b is a schematic diagram illustrating determining a composite sub-image according to some embodiments of the present disclosure; and FIG. 10c is a schematic diagram illustrating determining a target image according to some embodiments of the present disclosure. In some embodiments, a process 900 may be implemented on at least one machine, each machine including at least one processor and a storage device for ultrasound imaging. As shown in FIG. 9, the process 900 may include one or more of the following operations.

Operation 910, obtaining a plurality of original images produced based on a plurality of emissions of ultrasound waves, emission angles, or emission positions corresponding to the plurality of emissions of ultrasound waves being different. More information about the original image and emission of the ultrasound wave can be referred to FIG. 3 and the related descriptions thereof.

In some embodiments, a system for ultrasound imaging may obtain one or more original images using an ultrasound imaging device based on one or more emissions of ultrasound waves. One original image may be obtained by one emission of ultrasound waves one time. Each emission of ultrasound waves has a different emission angle or an emission position.

Operation 920, for each of the plurality of original images, determining a plurality of weighted original images based on a plurality of first weight images. More information about the first weight image and the weighted original image can be referred to FIG. 3 and the related descriptions thereof.

In some embodiments, for each of the plurality of original images, the system for ultrasound imaging may obtain the plurality of weighted original images by multiplying the plurality of first weight images with the plurality of original images. For example, as shown in FIG. 10a. for an original image 1, the system for ultrasound imaging may multiply a first weight image 1, a first weight image 2, . . . , and a first weight image n with the original image 1 to obtain a weighted original image 1, a weighted original image 2, . . . , and a weighted original image n, with n denoting a count of the first weight image.

Operation 930, for each of the plurality of first weight images, determining a composite sub-image based on weighted original images corresponding to the first weight image. More information about the composite sub-image can be referred to FIG. 3 and the related descriptions thereof.

In some embodiments, for each of the plurality of first weight images, the system for ultrasound imaging may perform coherent compounding on a plurality of weighted original images corresponding to the first weight image to obtain a composite sub-image corresponding to the first weight image. For example, as shown in FIG. 10b, for the first weight image 1, the system for ultrasound imaging may perform the coherent compounding on the weighted original image 1 corresponding to the original image 1, the weighted original image 1 corresponding to the original image 2, . . . , and the weighted original image 1 corresponding to the original image m to obtain a composite sub-image 1 corresponding to the first weight image 1, with m denoting a count of the original image.

Operation 940, determining a target image based on a plurality of composite sub-images corresponding to the plurality of first weight images. More information about the target image can be referred to FIG. 3 and the related descriptions thereof.

In some embodiments, the system for ultrasound imaging may perform spatial composite processing on the plurality of composite sub-images corresponding to the plurality of first weight images to obtain the target image. For example, as shown in FIG. 10c, the system for ultrasound imaging may perform the spatial composite processing on the composite sub-image 1 corresponding to the first weight image 1, a composite sub-image 2 corresponding to the first weight image 2, . . . , and a composite sub-image n corresponding to the first weight image n to obtain the target image, with n denoting the count of the first weight image.

Some embodiments of the present disclosure further provide a non-transitory computer-readable storage medium, the storage medium comprising at least one set of computer instructions. When executed by at least one processor of a computer device, the at least one set of instructions directs the at least one processor to obtain a plurality of original images produced based on a plurality of emissions of ultrasound waves, and for each original image of the plurality of original images, determine a weight dataset corresponding to each original image. The at least one set of instructions may further instruct the at least one processor to determine a composite sub-image corresponding to the each weight data subset based on the plurality of original images and a plurality of pieces of first weight information corresponding to the each weight data subset, and determine a target image based on a plurality of composite sub-images corresponding to a plurality of weight data subsets in the weight dataset.

In addition, certain features, structures, or characteristics of one or more embodiments of the present specification may be suitably combined.

Some embodiments use numbers to describe the number of components and attributes, and it should be understood that such numbers used in the description of the embodiments are modified in some examples by the modifiers “about”, “approximately”, or “substantially”. Unless otherwise noted, the terms “about,” “approximate,” or “substantially” indicate that a ±20% variation in the stated number is allowed. Correspondingly, in some embodiments, the numerical parameters used in the present disclosure and claims are approximations, which can change depending on the desired characteristics of individual embodiments. In some embodiments, the numerical parameters should take into account the specified number of valid digits and employ general place-keeping. While the numerical domains and parameters used to confirm the breadth of their ranges in some embodiments of the present disclosure are approximations, in specific embodiments, such values are set to be as precise as practicable.

In the event of any inconsistency or conflict between the descriptions, definitions, and/or use of terminology in the materials cited in the present disclosure and those described in the present disclosure, the descriptions, definitions, and/or use of terminology in the present disclosure shall prevail.

What is claimed is:

1. A method for ultrasound imaging, implemented on a computing device having at least one processor and at least one storage device, the method comprising:
obtaining a plurality of original images produced based on a plurality of emissions of ultrasound waves, emission angles, or emission positions corresponding to the plurality of emissions of ultrasound waves being different;
for each original image of the plurality of original images, determining a weight dataset corresponding to the each original image, the weight dataset including a plurality of weight data subsets, a count of the plurality of weight data subsets for each of the plurality of original images being the same;
for each weight data subset in the weight dataset, determining a composite sub-image corresponding to the each weight data subset based on the plurality of original images and a plurality of pieces of first weight information corresponding to the each weight data subset; and
determining a target image based on a plurality of composite sub-images corresponding to the plurality of weight data subsets in the weight dataset.

2. The method of claim 1, wherein at least two pieces of first weight information corresponding to a same weight data subset among at least two of the plurality of weight datasets are the same.

3. The method of claim 1, wherein the first weight information corresponding to the each original image includes a weight value corresponding to each position in the each original image.

4. The method of claim 1, wherein the for each weight data subset in the weight dataset, determining a composite sub-image corresponding to the each weight data subset based on the plurality of original images and a plurality of pieces of first weight information corresponding to the each weight data subset includes:
for each weight data subset in the weight dataset, determining a plurality of weighted original images based on the plurality of original images and the plurality of pieces of first weight information corresponding to the each weight data subset; and
determining the composite sub-image corresponding to the each weight data subset by performing coherent compounding on the plurality of weighted original images.

5. The method of claim 1, wherein the determining a target image based on a plurality of composite sub-images corresponding to the plurality of weight data subsets in the weight dataset includes:
generating a processed composite sub-image by performing an envelope detection processing on each composite sub-image;
for the each composite sub-image, determining second weight information by performing quality analysis on the each composite sub-image or the processed composite sub-image corresponding to the each composite sub-image; and
generating the target image based on the processed composite sub-image and the second weight information corresponding to the each composite sub-image.

6. The method of claim 1, wherein the first weight information is denoted by a first weight image, the first weight image including one or more weighted regions, and weighted regions of different first weight images being different.

7. The method of claim 1 wherein:
in response to the ultrasound waves being wide beams, the first weight information is determined such that each of the plurality of composite sub-images is a complete image, or a combination of the plurality of composite sub-images is a complete image.

8. The method of claim 7, wherein the first weight information is denoted by a first weight image, the first weight image including one or more weighted regions, the one or more weighted regions including a high-weighted region, different high-weighted regions in different first weight images have different positions or widths in a lateral direction of the first weight image.

9. The method of claim 1, wherein:
in response to the ultrasound waves being focused ultrasound beams, the first weight information is represented by a first weight image, the first weight image including one or more weighted regions, the first weight image is determined such that a width of the one or more weighted regions of the first weight image at a focal position of the focused ultrasound beams is narrower than that at other positions.

10. The method of claim 1, wherein:
in response to the ultrasound waves being focused ultrasound beams, the first weight information is represented by a first weight image, the first weight image is determined such that each of the plurality of composite sub-images includes an hourglass-type image region, or a combination of the plurality of composite sub-images is a complete image.

11. The method of claim 9, wherein the one or more weighted regions of the first weight image include a high-weighted region, and locations of high-weighted regions of different first weight images are different.

12. The method of claim 1, wherein the obtaining a plurality of original images produced based on a plurality of emissions of ultrasound waves includes:
determining a stage scanning feature based on a stage scanning result of emitted ultrasound waves by using a scanning feature extraction layer of a parameter prediction model, the parameter prediction model being a machine learning model; and
determining, by using a parameter prediction layer of the parameter prediction model, subsequent emission parameters and a count of subsequent emissions of the ultrasound waves based on, at least one of a historical count of emissions of emitted ultrasound waves, historical emission parameters of the emitted ultrasound waves, the stage scanning feature or a standard image.

13. The method of claim 1, further comprising:
determining the first weight information based on at least one of a type of the ultrasound waves, a count of the plurality of weight data subsets, the plurality of original images or parameters corresponding to the plurality of original images.

14. The method of claim 1, further comprising:
determining the first weight information using a first weight prediction model based on at least one of a type of the ultrasound waves, a count of the plurality of weight data subsets dataset emission parameters of the plurality of emissions of ultrasound waves, the plurality of original images, or parameters corresponding to the plurality of original images, the first weight prediction model being a machine learning model.

15. The method of claim 1, wherein the determining a target image based on a plurality of composite sub-images corresponding to the plurality of weight data subsets in the weight dataset includes:
determining the target image based on the plurality of composite sub-images and second weight information, the second weight information being determined based on the plurality of composite sub-images using a second weight prediction model, the second weight prediction model being a machine learning model.

16. The method of claim 15, wherein the determining the second weight information further includes:
dividing each composite sub-image into a plurality of sub-regions, and determining sub-image quality of each sub-region of the plurality of sub-regions, respectively, and determining sub-weight information of the each sub-region based on the sub-image quality; and
determining the second weight information based on the sub-weight information.

17. The method of claim 15, wherein the second weight information includes third weight information and fourth weight information, and the second weight information is determined by:
applying a low-pass filter or a speckle smoothing filter to the plurality of composite sub-images to generate an approximate image;
generating a detailed image based on the plurality of composite sub-images and the approximate image;
determining the third weight information and the fourth weight information using the second weight prediction model based on the approximate image and the detailed image, the third weight information being applied to the approximate image and the fourth weight information being applied to the detailed image.

18. A method for ultrasound imaging, implemented on a computing device having at least one processor and at least one storage device, the method comprising:
obtaining a plurality of original images produced based on a plurality of emissions of ultrasound waves, emission angles or emission positions corresponding to the plurality of emissions of ultrasound waves being different;
for each of the plurality of original images, determining a plurality of weighted original images based on a plurality of first weight images;
for each of the plurality of first weight images, determining a composite sub-image based on weighted original images corresponding to the first weight image; and
determining a target image based on a plurality of composite sub-images corresponding to the plurality of first weight images.

19. A system for ultrasound imaging, comprising:
at least one storage device including a set of instructions; and
at least one processor configured to communicate with the at least one storage device, wherein when executing the set of instructions, the at least one processor is configured to direct the system to perform operations including:
emitting a plurality of ultrasound waves, emission angles or emission positions corresponding to the plurality of emissions of ultrasound waves being different;
obtaining a plurality of original images produced based on a plurality of emissions of ultrasound waves;
for each original image of the plurality of original images, determining a weight dataset corresponding to the each original image, the weight dataset including first weight information, the first weight information including a plurality of pieces of first weight information, counts of the plurality of pieces of first weight information for the plurality of original images being the same;
for each weight data subset in the weight dataset, determining a composite sub-image corresponding to the each weight data subset based on the plurality of original images and a plurality of pieces of first weight information corresponding to the each weight data subset; and
determining a target image based on a plurality of composite sub-images corresponding to a plurality of weight data subsets in the weight dataset.

20. A non-transitory computer-readable medium, comprising at least one set of instructions, wherein when executed by at least one processor of a computer device, the at least one set of instructions directs the at least one processor to perform the method of claim 1.
